(19) United States
(12) Patent Application Publication
(10) Pub. No.: US 2026/0071183 A1
(43) Pub. Date: Mar. 12, 2026

(54) COMPOSITIONS AND METHODS RELATING TO ADIPOCYTE PRODUCTION

(71) Applicant: BaconBio LLC, New York, NY (US)

(72) Inventors: Samuel Budoff, Denver, CO (US); Avery Kravitz, Brooklyn, NY (US); Yipeng Wang, Pittsburgh, PA (US); Stanley Wang, New York, NY (US)

(73) Assignee: BaconBio LLC, New York, NY (US)

(21) Appl. No.: 19/122,929

(22) PCT Filed: Oct. 23, 2023
(86) PCT No.: PCT/US2023/035711

§ 371 Date: Apr. 21, 2025

Related U.S. Application Data
(60) Provisional application No. 63/418,236, filed on Oct. 21, 2022.

(51) Int. Cl. C12N 5/077; A23J 3/22; C12N 5/00; C12N 9/52

(52) U.S. Cl. CPC C12N 5/0653; A23J 3/227; C12N 5/0062; C12N 5/0081; C12N 9/52; C12Y 304/24003; C12N 2500/34; C12N 2500/99; C12N 2506/13; C12N 2513/00

(57) ABSTRACT

Provided in this disclosure is a method of cyclical production of fat cells. The method may include the steps of dedifferentiation of adipocytes into dedifferentiated fat (DFAT) cells, permitting the DFAT cells to proliferate, and re-differentiating (differentiating) the proliferated DFAT cells into adipocytes. Additionally, provided in this disclosure are methods of isolating DFAT cells adipose from a range of species. Also provided in this disclosure are fat-producing cells and method of creating the same. Such cells can be used for a variety of purposes and products, including imitation meat products.

### FIELD

The present disclosure relates in part to the field of cellular agriculture. In particular embodiments, the present disclosure relates to methods of cyclical production of fat cells. More specifically, embodiments of the disclosure relate to cyclical dedifferentiation of adipocytes into DFAT cells and subsequent re-differentiation of the DFAT cells. The systems and methods disclosed herein can be particularly useful for the creation of palatable imitation meat products. The present disclosure also relates to other commercially valuable areas including, but not limited to, aromatics and flavorings, cell-therapy, cosmetics, drug discovery, industrial manufacturing, regenerative medicine, renewable energy, and veterinary medicine.

### BACKGROUND

Over 50% of Americans aged 24-39 now describe themselves as flexitarians, such that a large and growing body of consumers are seeking meat alternatives to support more sustainable, environmentally friendly, and ethical diets. However, most consumers-flexitarian or otherwise have been unwilling to fully embrace meat alternatives, such as plant-based meat alternatives (PBMAs), because, for example, the differences between conventional meat products and their alternatives remain noticeable, and the long ingredients lists in highly processed meat alternatives are a consumer deterrent.

The perceived taste/texture, cost, health, and sustainability are key drivers of dietary decision-making among PBMA customers. Importantly, while most PBMAs cater to a vegan customer, vegans make up only 2% of such sales, with the remaining 98% being people who want to consume fewer meat products but who may be unwilling to transition completely to a vegan diet. As such, PBMAs still make up a mere 1.5% of meat sales.

Cultured meat, also referred to as in vitro or lab-grown meat, is a promising technology, as it can reduce the greenhouse gas emissions, land use, and water use required for meat production. This technology is still in its infancy, and while a number of companies have demonstrated pilot products, cost and scale-up challenges represent barriers to commercialization. In addition, today's emerging cultured meat products rely on fetal bovine serum (FBS) as a growth medium during production. Because FBS must be harvested directly from the fetus of a slaughtered cow, there are steep costs associated with the extensive quality control procedures and regulatory oversight necessary to prevent contamination and ensure ethical compliance.

Key flavor differences between conventional meat products and their alternatives are driven by fat content. Plant-based meat companies, for example, have attempted to use complex cocktails of ingredients to mimic fat; however, this approach falls short of authentically emulating the desired texture/taste, and also contributes to extensive ingredients lists. Indeed, flavor additives make up around 3-10% of PBMA products, and attempts to emulate fat tissue are often recognized as the biggest challenge faced by PBMA formulators.

Thus, there is a need for new methods of enhancing various properties of PBMAs and other meat alternatives through the addition of improved alternatives to animal fat tissue. The present disclosure satisfies this need by providing fat-producing cells from various animal species that can be incorporated into alternative meat products to generate convincing high quality meat alternatives with shorter ingredients lists and taste/texture profiles that are consistent with traditional meat products. The present disclosure satisfies this need and provides other advantages as well.

### SUMMARY

In one aspect, the present disclosure provides a method of cyclical production of fat cells, the method comprising dedifferentiating a population of mature adipocytes to create a first population of dedifferentiated fat cells (DFAT cells). In some embodiments, the method comprises culturing the first population of DFAT cells in a growth media until reaching a first target biomass. In certain embodiments, the first target biomass is then subcultured into one or more subculture populations of DFAT cells. The one or more subculture populations can be re-differentiated, e.g., to form re-differentiated mature adipocytes. In some embodiments, the method comprises re-differentiating one or more DAFT cells within the first target biomass into re-differentiated mature adipocytes.

The re-differentiated mature adipocytes can be separated into a first portion and a second portion. In such embodiments, the method further comprises dedifferentiating the first portion of re-differentiated mature adipocytes to create a second generation of DFAT cells and culturing the second portion of the re-differentiated mature adipocytes in culture media to accumulate a target lipid mass. In particular embodiments, the target lipid accumulation is about 90% of the cell volume. In particular embodiments, the target lipid accumulation will be as low as 20% of the cell volume. Those skilled in the art will appreciate that target lipid accumulation depends on accumulation time and concentration of feed reagents, with capacities of cells for lipid accumulation of about 15%, 20%, 25%, 30%, 35%, 40%, 45%, 50%, 55%, 60%, 65%, 70%, 75%, 80%, 85%, 95%, or more of the cell volume with the specific value being determined by one skilled in the art. In particular embodiments, the culture medium comprises glucose at a concentration of from about 4% glucose (i.e., 4 g/L glucose) to about 20% glucose (i.e., 20 g/L glucose), or of from about 4% to about 15% glucose, or of from about 4% to about 10% glucose, or of from about 4% to about 8% glucose, or about 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, or 19%, glucose (g/L). In some embodiments, the culture medium comprises 4.5% glucose (g/L).

In certain embodiments, a re-differentiation medium is used. Re-differentiation media can comprise, e.g., insulin, 3-isobutyl-1-methylxanthine, insulin-transferrin-selenium-X supplement, indomethacin, dexamethasone, rosiglitazone, biotin, pantothenate, a PPARγ agonist, sodium oleate, wheat germ agglutinin, or any combination thereof.

In some embodiments, the method further comprises: i) dedifferentiating at least a portion of the re-differentiated mature adipocytes to create a second generation of DFAT cells; ii) culturing the second generation of DFAT cells in a growth medium until a second target biomass is obtained; iii) subculturing the second target biomass into a second generation of sub-cultured DFAT cells; and iv) re-differentiating the second generation of sub-cultured DFAT cells into a second generation of re-differentiated mature adipocytes.

The method can further comprise cyclically repeating the steps of one or more of dedifferentiating, culturing, subculturing, and re-differentiating for a total of n cycles. In some embodiments, the step of dedifferentiating in each cycle is performed on the re-differentiated mature adipocytes that were re-differentiated in the immediately preceding cycle. In certain embodiments, n is equal to the number of cycles during which the re-differentiated mature adipocytes maintain the ability to proliferate and/or maintain the ability to synthesize and store fat. In some embodiments, n is equal to the number of cycles during which the ability to proliferate and/or maintain the ability to synthesize and store fat of the re-differentiated mature adipocytes has not declined by more than about, e.g., 5%, 10%, 15%, 20%, 25%, 30%, 35%, 40%, 45%, 50%, 55%, 60%, 65%, 70%, 75%, 80%, or more as determined by one skilled in the art for their target use case.

In particular embodiments, the population of mature adipocytes are obtained from adipose cells or a tissue explant that comprises adipose tissue, i.e., by isolating the population of mature adipocytes from the adipose cells or tissue explant. The step of isolating the population of mature adipocytes can comprise adding the adipose cells or tissue explant to a digestion buffer in a container (i.e. a cell culture container). In certain embodiments, the adipose cells or tissue explants are incubated in the digestion buffer for, e.g., at least about 30 minutes. In some embodiments, following the incubation, the adipose cells and digestion buffer are centrifuged to create a first floating adipocyte layer (FAL) above the digestion buffer and a pelleted tissue at the bottom of the container. In particular embodiments, the digestion buffer and pelleted tissue are removed from the container, and the first FAL is suspended in growth media. The first FAL and growth media can then be centrifuged to create a second FAL and a second pelleted tissue. In some embodiments, the step of centrifuging is performed without filtration. In particular embodiments, the digestion buffer and second pelleted tissue are removed from the container, and the second FAL is suspended in growth media, wherein the second FAL comprises the mature adipocytes.

In particular embodiments, the adipose cells are added to the digestion buffer at a concentration of about I gram of adipose cells or tissue explant per 3 ml of digestion buffer. In some embodiments, the adipose cells are added to the digestion buffer at a concentration of about 0.1, 0.5, 1.5, 2, 2.5, 3, or more grams of adipose cells or tissue explant per 3 ml of digestion buffer. In particular embodiments, the digestion buffer comprises a collagenase enzyme. In some embodiments, the digestion buffer comprises 0.2%-1% collagenase, at least 0.2% collagenase, or up to 1% collagenase. In particular embodiments, the digestion buffer comprises a collagenase derived from microbial fermentation. The collagenase used in the digestion buffer can be selected based on the species of the source cells to be digested. In some embodiments, the source cells are mammalian cells and the collagenase used is collagenase I, II, or IV or a collagenase enzyme having a similar activity level to that of collagenase IV. In some embodiments, the source cells are avian cells and the collagenase used is collagenase IV or a collagenase enzyme having a similar activity level to that of collagenase IV. Importantly, collagenase IV can be used in the digestion buffer for digestion of mammalian cells and/or avian cells. In particular embodiments, the collagenase comprises collagenase IV (e.g., collagenase IV from Clostridium histolyticum) and variants thereof having similar enzymatic activity. In some embodiments, the digestion buffer comprises at least 0.2% collagenase IV. In some embodiments, the digestion buffer comprises up to 1% collagenase IV.

In particular embodiments, the tissue explant comprises subcutaneous fat extracted from a subject. The subject can be, e.g., a mammal, a bird, a fish, a reptile, a crustacean, or an amphibian. In some embodiments, the tissue explant comprises subcutaneous fat extracted from a plurality of subjects. In some embodiments, the subjects are the same species. In some embodiments, the subjects are different species.

In certain embodiments, the adipose cells comprise primary mammalian cells, primary avian cells, primary fish cells, primary reptilian cells, primary crustacean cells, primary amphibian cells, or a combination thereof. In some embodiments, the adipose cells do not comprise or are not derived from any one or more of pluripotent stem cells, embryonic stem cells, or undifferentiated stem cells.

In some embodiments, the step of dedifferentiating the adipose cells comprises: i) seeding the mature adipocytes in a dedifferentiation container with a volume of growth media sufficient to completely fill the container; ii) closing the dedifferentiation container to create a hypoxic environment; iii) inverting the dedifferentiation container; iv) incubating cells within the inverted dedifferentiation container; v) permitting the mature adipocytes to adhere to the ceiling surface of the dedifferentiation container; and vi) allowing the mature adipocytes to dedifferentiate into DFAT cells. In some embodiments, the cells are incubated in the inverted dedifferentiation container for about 10 days, or about 3, 4, 5, 6, 7, 8, 9, 11, 12, 13, 14 or more days. One skilled in the art will appreciate that the process efficiency near 10 days as here disclosed is most efficient but modifications to environmental conditions such as incubation temperature, dissolved gasses, etc. may allow incubation times at the extremes or possibly beyond those here listed to work. In some embodiments, the cells are incubated in the inverted dedifferentiation container for a number of days sufficient to observe a fibroblastic morphology accompanying loss of central lipid accumulation.

In some embodiments, the step of re-differentiating a population of DFAT cells, e.g., re-differentiating one or more subculture populations of DFAT cells or a population of DFAT cells within a first target biomass, into mature adipocytes comprises contacting the one or more subculture populations of DFAT cells or the DAFT cells within the first target biomass with an adipocyte differentiating-promoting agent such as insulin, 3-isobutyl-1-methylxanthine, insulin-transferrin-selenium-X supplement, indomethacin, dexamethasone, rosiglitazone, biotin, pantothenate, a PPARγ agonist, sodium oleate, wheat germ agglutinin, or any combination thereof.

In some embodiments, the step of re-differentiating the one or more subculture populations of DFAT cells or the DFAT cells within a first target biomass can comprise manipulating one or more parameters to preferentially encourage the formation of re-differentiated mature adipocytes. Such parameters can comprise, e.g., the presence or nature of a coating to an interior surface of the container (i.e. culture container) containing the cells, one or more mechanical properties of a culture environment (i.e. the elasticity of the culture environment as controllable with for example hydrogels), growth media density, growth media osmolarity, growth media temperature, factors that encourage DFAT cell proliferation rather than lipid production, and agents to control the differentiation of non-adipocyte cells.

In particular embodiments, the first target biomass is reached upon achieving at least about 70% confluence of DFAT cells. In some embodiments, the first target biomass is reached upon achieving at least about 50%, 60%, 65%, 75%, 80%, 85%, or more confluence of DFAT cells.

In particular embodiments, the growth medium as used in various methods of this disclosure comprises only food-safe components. In some embodiments, the growth media is serum-free. In some embodiments, the growth media does not comprise fetal bovine serum or fetal calf serum. In some embodiments, the growth medium is plant-based, e.g., a medium as described in publications such as Yamanaka 2023, the entire disclosure of which is herein incorporated by reference, where the growth medium is a formulation comprising canola oil and/or other growth factors. In some embodiments, the animal-product free formulation is obtained from a commercial supplier.

In some embodiments, the step of culturing a first population of DFAT cells and/or subculturing a first target biomass comprises culturing the cells in a growth media until a first target biomass or a second target biomass, respectively, is achieved (i.e. a desired level of confluency in the culture container. In some embodiments, the step of culturing a first population of DFAT cells and/or subculturing a first target biomass comprises seeding the growth media with a concentration of cells at least 5000-10,000 cells/cm² and incubating until the cells reach at least 70% confluence. In some embodiments, the growth medium is seeded with a concentration of at least, e.g., 2,000; 3,000; 4,000; 5,000; 6,000; 7,000; 8,000; 9,000; 10,000; 11,000; 12,000; cells/cm², and/or the cells are incubated until they reach about 50%, 55%, 60%, 65%, 75%, 80%, or more confluence. One skilled in the art will appreciate that theoretically greater or lower values than those presented are technically possible but may be inefficient under standard monolayer cell culture conditions. In some embodiments, 5000-10,000 cells/cm² are seeded. In some embodiments, 1000-5000 cells/cm² are seeded. In some embodiments, 10,000-12,000 cells/cm² are seeded.

In another aspect, the present disclosure provides a fat-producing cell line created by any of the various methods disclosed herein. The fat-producing cell line can comprise, e.g., a total lipid accumulation of at least 80% of an intracellular volume when the cell line is at least, e.g., 4 months old. In some embodiments, the fat-producing cell line comprises DFAT cells produced using the de-differentiating methods provided in this disclosure.

Additional aspects of the present disclosure include a cell line of re-differentiated fat-producing cells created by any of the various methods disclosed herein, wherein the cell line is configured to survive in a suspension culture.

Another aspect includes an imitation meat product for consumption comprising fat cells created by any of the methods disclosed herein, e.g., an effective amount of fat cells to produce a desired flavor and/or texture profile, and an alternative meat source, e.g., an effective amount of an alternative meat source. In some embodiments, the created fat cells represent up to about 5% of the total volume or weight of imitation meat product (or up to about 1%, 2%, 3%, 4%, 6%, 7%, 8%, or more of the total volume or weight). In some embodiments, the alternative meat source represents at least about 10% of the total volume or weight of the meat product (or at least about 15%, 20%, 25%, 30%, 35%, 40%, 45% 50%, or more of the total volume or weight). In certain embodiments, the alternative meat source comprises cultured meat, a plant-based meat alternative, insect-derived proteins, fermentation-derived products, fungal proteins, or a combination thereof.

In yet another aspect, the present disclosure relates to a method of creating an imitation meat product for consumption. In various embodiments, the method comprises i) dedifferentiating a population of mature adipocytes to create a first population of DFAT cells; ii) culturing the first population of DFAT cells in a growth media until reaching a first target biomass; iii) subculturing the first target biomass into one or more subculture populations of DFAT cells; iv) re-differentiating the one or more subculture populations of DFAT cells into re-differentiated mature adipocytes; and v) combining the re-differentiated mature adipocytes with an effective amount of an alternative meat source to create the imitation meat product. In some embodiments, the step of dedifferentiating the population of mature adipocytes to create a first population of DFAT cells comprises digesting the population of mature adipocytes with collagenase IV or a collagenase having similar enzymatic activity thereto. In certain embodiments, the population of mature adipocytes comprises at least one of mammalian or avian cells.

An additional aspect of the present disclosure includes an in vitro method of creating fat-producing cell lines, wherein the fat-producing cell lines comprise DFAT cells. In some embodiments, the method comprises dedifferentiating a population of mature adipocytes to create a first population of DFAT cells, wherein this step comprises digesting the population of mature adipocytes with collagenase IV or a collagenase having similar enzymatic activity thereto. The method can further include culturing the first population of DFAT cells in a growth media until a first target biomass is reached. In some embodiments, the growth media comprises only food-safe components. In certain embodiments, the method comprises subculturing the first target biomass into one or more subculture populations of DFAT cells. The method can further include re-differentiating the one or more subculture populations of DFAT cells into re-differentiated mature adipocytes. In certain embodiments, the population of mature adipocytes comprises at least one of mammalian or avian cells.

In another aspect, the disclosure includes a transduction-free method of renewing primary cells. In embodiments, the method includes i) dedifferentiating a population of mature adipocytes to create a first population of DFAT cells; ii) culturing the first population of DFAT cells in a growth media until reaching a first target biomass; iii) subculturing the first target biomass into one or more subculture populations of DFAT cells; and iv) re-differentiating the one or more subculture populations of DFAT cells into re-differentiated mature adipocytes. In some embodiments, the step of dedifferentiating the population of mature adipocytes to create a first population of DFAT cells comprises digesting the population of mature adipocytes with collagenase IV or a collagenase having similar enzymatic activity thereto. In certain embodiments, the population of mature adipocytes comprises at least one of mammalian or avian cells.

One aspect of the present disclosure relates to a method of reducing senescence in a cell line comprised of primary fat cells. In various embodiments, the method comprises: i) dedifferentiating a population of primary mature adipocytes to create a first population of DFAT cells; ii) culturing the first population of DFAT cells in a growth media until reaching a first target biomass; iii) subculturing the first target biomass into one or more subculture populations ofDFAT cells; and iv) re-differentiating the one or more subculture populations of DFAT cells into re-differentiated mature adipocytes. In some embodiments, the step of dedifferentiating the population of primary mature adipocytes to create a first population of DFAT cells comprises digesting the population primary of mature adipocytes with collagenase IV or a collagenase having similar enzymatic activity thereto. In certain embodiments, the population of primary mature adipocytes comprises at least one of mammalian or avian cells.

An additional aspect of the present invention relates to a commercially scalable cell line for producing consumable, cultured animal fat. The cell line can comprise re-differentiated fat-producing cells created by any of the various methods disclosed herein. In some embodiments, the consumable, cultured animal fat is produced using the cell line in a commercial-scale bioreactor.

An additional aspect includes a method of creating at least two populations of DFAT cells. One skilled in the art will recognize that clonal populations of daughter cells can be readily achieved, and these populations can be remixed to create a heterogenous population of DFAT cells originating from a single biopsy. Alternatively, bulk or clonal DFAT populations from several biopsies, either on a single animal or two or more animals, can be combined to create a heterogenous population. In various embodiments, the method comprises obtaining adipose cells or a tissue explant that comprises adipose tissue, and isolating a population of mature adipocytes from the adipose cells or tissue explant. In some embodiments, the step of isolating the population of mature adipocytes comprises: i) adding the adipose cells or tissue explant to a digestion buffer in a container, wherein the digestion buffer comprises a collagenase; ii) incubating the adipose cells or tissue explant in the digestion buffer until homogenous digestion is achieved (for example, for at least about 30 minutes); iii) centrifuging the adipose cells and digestion buffer to create a FAL above the digestion buffer and a pelleted tissue at the bottom of the container, wherein this step of centrifuging does not comprise filtration; iv) removing the digestion buffer and pelleted tissue from the container; v) suspending the first FAL in growth media; vi) centrifuging the first FAL and growth media to create a second FAL and a second pelleted tissue; vii) removing the digestion buffer and the second pelleted tissue from the container; and viii) suspending the second FAL in growth media, wherein the second FAL comprises the mature adipocytes.

In some embodiments, the method further comprises: ix) seeding the mature adipocytes in a dedifferentiation container with a volume of growth media sufficient to completely fill the container; x) closing the dedifferentiation container to create a hypoxic environment; xi) inverting the dedifferentiation container; xii) incubating the inverted dedifferentiation container for about 10 days; xiii) permitting the mature adipocytes to adhere to a celling surface of the dedifferentiation container; and xiv) allowing the mature adipocytes to dedifferentiate into the at least two heterogenous populations of DFAT cells.

Other objects and advantages of this invention will become readily apparent from the ensuing description.

### BRIEF DESCRIPTION OF THE DRAWINGS

These figures are intended to be illustrative, not limiting. Although the Examples of the disclosure are generally described in the context of these figures, it should be understood that it is not intended to limit the scope of the disclosure to these particular Examples.

FIG. 1 provides a schematic of a protocol for dedifferentiation of mature fat cells into DFAT cells under one embodiment of the present disclosure, followed by cycling of the DFAT cells to mature adipocytes and back. Cycling can, but does not have to, include an accumulation phase following the re-differentiation of DFAT cells. Likewise, proliferation of biomass by techniques such as passaging and expanding can, but do not need to, be performed during a given cycle round. Finally, while the schematic demonstrates only a single petri-dish-like vessel at any step, any suitable cell culture system as used by those skilled in the art are equally appropriate.

FIG. 2 shows an exemplary historical model developed by Sugihara et al. (1986) demonstrating how TAG-filled vesicles are transferred from mother to daughter cells, and the relationship between the differentiation and TAG accumulating phenotypic stages.

FIGS. 3A-3B are micrograph images of chicken adipose tissue following digestion with collagenase I or II. These micrograph images show that the solution contains only lysed cells, including in some cases large TAG aggregations, demonstrating that traditional DFAT isolation methods developed using mammalian cells do not allow the successful isolation of avian DFAT cells. FIG. 3A provides a representative light microscopy photograph showing unsuccessful isolation of adipocytes from chicken fat tissue when collagenase I was utilized in the digestion buffer and filtration was employed with centrifugation. FIG. 3B provides a representative light microscopy photograph showing unsuccessful isolation of adipocytes from chicken fat tissue when collagenase II was utilized, and centrifugation was performed without filtration. Scale bar in FIG. 3A image represents 100 μm and is applicable to the image in FIG. 3B as well (same magnification).

FIGS. 4A-4F present pictorial micrographs showing avian adipocytes and DFAT cells derived using the methods according to certain aspects and embodiments of this disclosure. FIG. 4A) Dissociated adipocytes viewed in floating suspension under brightfield illumination. FIG. 4B) DFAT cells derived from the mature adipocytes in FIG. 4A stained with the lipid dye Nile Red. FIG. 4C) Enlarged view of Nile Red stained DFAT cell from FIG. 4B wherein Nile Red staining highlights fibroblastic pseudopodia and ‘loculus-dividing’ lipid droplets and dividing nucleus.. 4D) Brightfield illumination of DFAT cells derived from the mature adipocytes in FIG. 4A, the same imaging region as FIG. 4B. FIG. 4E) Derived DFAT cells from the mature adipocytes in FIG. 4A stained with the nucleic acid dye Hoechst 33432. FIG. 4F) Enlarged view of a single DFAT cell undergoing mitosis, wherein Hoechst 33432 nuclear staining highlighting mitosis as nuclear material shuttling into splitting nuclei, indicating this cell is in anaphase or telophase of mitosis. Scale bars in images represent 50 μm (FIGS. 4A-4B, 4D-4E) and 5 μm (FIGS. 4C, 4F).

FIGS. 5A-5C provide a sequential series of pictorial micrographs showing a timelapse of DFAT re-differentiation according to certain aspects and embodiments of this disclosure. FIG. 5A) Two days after the introduction of differentiation media. FIG. 5B) Five days after the introduction of differentiation media. FIG. 5C) Two weeks after the introduction of differentiation media. Scale bars in images represent 100 μm.

FIGS. 6A-6D show pictorial micrographs that demonstrate a timelapse of the DFAT cycling process according to certain aspects and embodiments of this disclosure. FIG. 6A) Two days after the introduction of differentiation media. Black arrow indicates a large lipid droplet. Differentiation media is then removed. FIG. 6B) Two days after the removal of differentiation media. Cells are then passaged. FIG. 6C) One day after passage and the re-introduction of differentiation media. FIG. 6D) Two days after the re-introduction of differentiation media. Lipid accumulation observed, as indicated by black arrow. Scale bars in images represent 100 μm.

FIGS. 7A-7D provide sequential images of pictorial micrographs to demonstrate the re-differentiation of DFAT and the subsequent induced return to multi-potent morphology according to certain aspects and embodiments of this disclosure. FIG. 7A) One day after seeding. Differentiation media is then introduced. FIG. 7B) Five days after the introduction of differentiation media. Arrow indicates a large lipid droplet. Cells are then passaged. FIG. 7C) One day after passage. Cells remain in growth media. FIG. 7D) Ten days after passage. Scale bars in images represent 100 μm.

FIG. 8 provide a cladogram and images of DFAT cells derived from various species, as observed on the ceiling of the de-differentiation container according to certain aspects and embodiments of this disclosure. FIG. 8 illustrates the substantial evolutionary space upon which the methods disclosed herein have been reduced to practice without modification. From top to bottom, as indicated, pictorial micrographs show Turkey derived DFAT, Chicken derived DFAT, Duck derived DFAT, Pigeon derived DFAT, Rabbit derived DFAT. Molecular relationships quantified in the cladogram were computed based on alignment of published 18s rRNA sequences. Conversion of molecular relationship to molecular clock time, in millions of years (MY), was based on a conservative coefficient, and these values are indicated above their respective branches. Scale bars in micrograph images represent 100 μm.

### DETAILED DESCRIPTION

### Abbreviations and Definitions

The following description recites various aspects and embodiments of the present methods and compositions. It is to be understood that the present invention may be embodied in various forms. No particular embodiment is intended to define the scope of the methods and compositions. Rather, the embodiments merely provide non-limiting examples of various methods and compositions that are at least included within the scope of the disclosed methods and compositions. Therefore, specific details disclosed herein are not to be interpreted as limiting, but rather as a basis for the claims and as a representative basis for teaching one skilled in the art to employ the present invention in any appropriate manner. The description is to be read from the perspective of one of ordinary skill in the art; therefore, information well known to the skilled artisan is not necessarily included.

The singular forms “a”, “an” and “the” include plural reference unless the context clearly dictates otherwise. The use of the word “a” or “an” when used in conjunction with the term “comprising” in the claims or the specification may mean “one,” but it is also consistent with the meaning of “one or more,” “at least one,” and “one or more than one.”

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C. As used herein, “and/or” refers to and encompasses any and all possible combinations of one or more of the associated listed items, as well as the lack of combinations where interpreted in the alternative (“or”).

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b, and c.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of up to 20 percent up or down (higher or lower), e.g., a value ±1%, ±2%, ±3%, ±4%, ±5%, ±6%, ±7%, ±8%, 9%, ±10%, ±11%, ±12%, ±13%, ±14%, ±15%, ±16%, ±17%, ±18%, ±19% or ±20% of the stated value.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the term “isolated” when used in reference to certain cells or adipocytes refers to cells that have been removed or disassociated from an organism, organ, tissue, or culture in which they were originally found or with which they were previously associated. In some embodiments, isolated cells or adipocytes are cells or adipocytes in suspension. In some embodiments, isolated cells or adipocytes are cells or adipocytes in a cell culture flask. In some embodiments, isolated cells or adipocytes are cultured cells or adipocytes. In some embodiments, isolated cells or adipocytes are a component of a larger mixture of cells including a tissue sample or a suspension with non-adipocyte cells. In some embodiments, cells or adipocytes are considered “isolated” when they are removed from the animal from which they are derived, such as in the case of a tissue explant. In some embodiments, the isolated cells or adipocytes can comprise at least one or a plurality of cells that are from a species capable of producing adipocytes and/or that natively comprises adipose tissue, e.g., one or more species chosen from mammals, birds, fish, reptiles, crustaceans, or amphibians. In some embodiments, the isolated cells or adipocytes comprise sheep cells, goat cells, horse cells, cow cells, human cells, monkey cells, mouse cells, rat cells, rabbit cells, canine cells, feline cells, porcine cells, chicken cells, turkey cells, duck cells, pigeon cells, guinea fowl cells, ostrich cells, or cells from another non-human animal. In non-food embodiments, the isolated cells or adipocytes may be at least one of human cells or non-human primate cells.

In some embodiments, adipose cells (also referred to as adipocytes or fat cells in this disclosure) are derived from a subject. The subject can comprise any vertebrate or invertebrate animal. In some embodiments, a subject comprises a mammal, a bird, a fish, a reptile, a crustacean, or an amphibian. In non-food embodiments, the subject may be human or non-human primate. In some embodiment, the adipocytes or adipose tissue comprises white, brown, or beige adipocytes, or a combination thereof. In some embodiment, the adipocytes or adipose tissue comprises visceral fat, subcutaneous fat, or a combination thereof.

As used herein, “growth medium” (also referred to in this disclosure as cell culture medium, culture medium, basal medium, and a basal culture medium) is a liquid designed to support the growth of cells. Cell culture media generally comprise an appropriate source of energy and compounds that regulate the cell cycle. A typical culture medium is composed of a complement of amino acids, vitamins, and inorganic salts. It may also contain one or more of glucose, serum as a source of growth factors, hormones, and attachment factors (e.g., fibronectin). In addition to nutrients, the medium also contains components that help maintain pH and osmolality. In some instances, growth medium may contain indicators such as phenol red, which can be used to monitor the state of the culture system. In some embodiments, growth medium may contain anti-biotic or anti-microbial agents. Growth medium may also contain specific ions related to other physical additives in the culture system, such as alginates or collagen fibrils used in 3-D culture systems. Growth medium may contain carrier particles in the case a suspension culture is performed.

The term “target biomass” as used in this disclosure refers to a desired amount of cells obtained through culturing methods, for example, using the methods provided in this disclosure to achieve a degree of confluency (cell number) within a container (i.e. cell culture container).

As used herein, the phrase “resultant fat-producing cell line(s)” refers to one or more cell lines produced using any one or more of the methods disclosed herein. In some embodiments, the resultant fat-producing cell line comprises one or more mature adipocytes that have been re-differentiated according to any of the various embodiments disclosed herein.

The phrase “alternative meat source” as described herein can comprise any source of a food protein product that does not comprise animal flesh obtained by livestock. The terms “alternate meat” and “alternative meat” can refer to food proteins that are suitable for animal consumption, human consumption, or a combination thereof. Exemplary alternative meat sources include cell-based proteins, plant-based proteins, insect-derived proteins, fermentation-derived proteins, fungal proteins, or a combination thereof. The phrase “alternative meat product,” “alternative meat,” and the like can be used interchangeably to refer to any product obtained from an alternative meat source. Exemplary alternative meat products include a cultured meat, a plant-based meat alternative (PBMA), insect-derived proteins, fermentation-derived products, fungal proteins, dairy-derived meat alternatives, egg-derived meat alternatives, gelatin-derived meat alternatives, keratin-derived meat alternatives, meat alternatives placed on or within a plant-based scaffolding, or a combination thereof.

### DFAT Cells

Aspects of the present disclosure are directed to the generation and culturing of dedifferentiated fat (DFAT) cells as well as to the production of adipocytes therefrom. In some aspects of the disclosure, DFAT cells are re-differentiated into mature adipocyte cells to create a resultant fat-producing cell line. In various embodiments, the present disclosure relates to resultant fat-producing cell lines, methods of producing the same, and methods of using such cell lines to produce adipocytes. Certain embodiments relate to use of a cell line, e.g., an avian cell line, or a primary culture to produce DFAT cells, adipocytes, and/or commercially viable animal fat.

The present disclosure provides methods for producing DFAT cells starting from adipose cells, or tissues comprising adipose cells. Adipose tissue or adipocytes from any of the source species described in this disclosure can be used as a source for the production of DFAT cells and to produce fat-producing cell lines therefrom. In particular embodiments, the cell lines are obtained from an avian species.

The DFAT cells produced using the methods provided in this disclosure can be used in any of a number of ways. For example, under specific conditions, DFAT cells can be induced to re-differentiate into any of a variety of cell types, including adipocytes, osteoblasts, cardiac muscle cells, pericytes, and/or other cell types important for tissue engineering. In therapeutic or veterinary contexts, DFAT cells may be injected into a human or animal patient with or without further in vitro differentiation (e.g., for cell therapy, as discussed below in this disclosure).

The fat-producing cell lines can be incorporated into a variety of products, e.g., into meat alternatives. The resultant fat-producing cell lines can be utilized in the production of an alternative meat product that comprises adipocytes from the fat-producing cells. In some embodiments, DFAT cells, or the fat-producing adipocytes derived therefrom using the methods provided in this disclosure, can be used to enhance the taste of alternative meat products and/or reduce the ingredient lists of such products. In some embodiments, DFAT cells, or the fat-producing adipocytes derived therefrom using the herein-disclosed methods, can be used for other commercially valuable pursuits including, but not limited to, those related to aromatics and flavorings, cell-therapy, cosmetics, drug discovery, industrial manufacturing, regenerative medicine, renewable energy, and veterinary medicine.

Certain aspects of the present disclosure are based in part of the innovation by the inventors that cycles of dedifferentiation and re-differentiation or trans-differentiation of adipocytes into DFAT cells into adipocytes or other cells could be used to continuously produce adipocytes or other cells in culture for extended periods of time without genetic manipulation of any of the cells. A benefit of DFAT cells is that they can be readily cultured for extended periods of time (i.e. passages). This stands in contrast with standard methods for producing induced pluripotent stem cells, which by definition require one to manipulate genetic factors in a terminally differentiated cell, such as a fibroblast, to achieve a stem-cell like phenotype. It also stands in contrast with methods of immortalizing primary cells, similarly by means of genetic manipulation, as can be done with myoblasts to expand the amount of biomass derivable from a given tissue sample.

DFAT cells have a number of properties that can be used to aid in their identification. For example, various molecular markers of multipotency are commonly upregulated in DFAT cells. Accordingly, in some embodiments, DFAT cells display statistically significance increases in, e.g., telomerase protein, telomerase transcripts, and/or telomerase enzymatic activity. In some embodiments, DFAT cells present one or more similarities, e.g., express similar molecular markers, to Stromal Vascular Cells (also known as Adipose-Derived Stem cells, ADSCs), the resident stem cells that give rise to adipocytes in vivo. In some embodiments, telomerase activity is greater in DFAT cells than in ADSCs. Without being bound by the following theory, it is believed that the elevated telomerase activity seen in DFAT cells renders the cells more resistant to senescence than other commonly pursued cell types. DFAT cells can also be identified in part by certain typical morphological properties, e.g., having a fibroblastic morphology, or having lost accumulated tri-acetyl glyceride (TAG).

Additionally, certain aspects of the present disclosure are based in part on the surprising discovery that traditional methods of producing DFAT cells from mammalian adipose cells or tissues, e.g., traditional methods involving collagenase I or collagenase II as described, e.g., in Yagi 2004, Wei 2013, Watson 2014, Jumabay 2015, Shah 2016, Pu 2017, Lu 2018, Cui 2018, Zang 2020, Alexanderson 2020, Rogal 2020, Schopow 2020, Cho 2020, Panella 2021, and Hendaway 2021; are not effective at producing avian DFAT cells.

In contrast, the methods described herein for producing DFAT cells, e.g., involving collagenase IV or other collagenase enzymes characterized by low tryptic activity, are effective at producing DFAT cells from avian sources as well as other sources, including mammalian cells.

Isolation and Dedifferentiation of Adipocytes into DFAT Cells

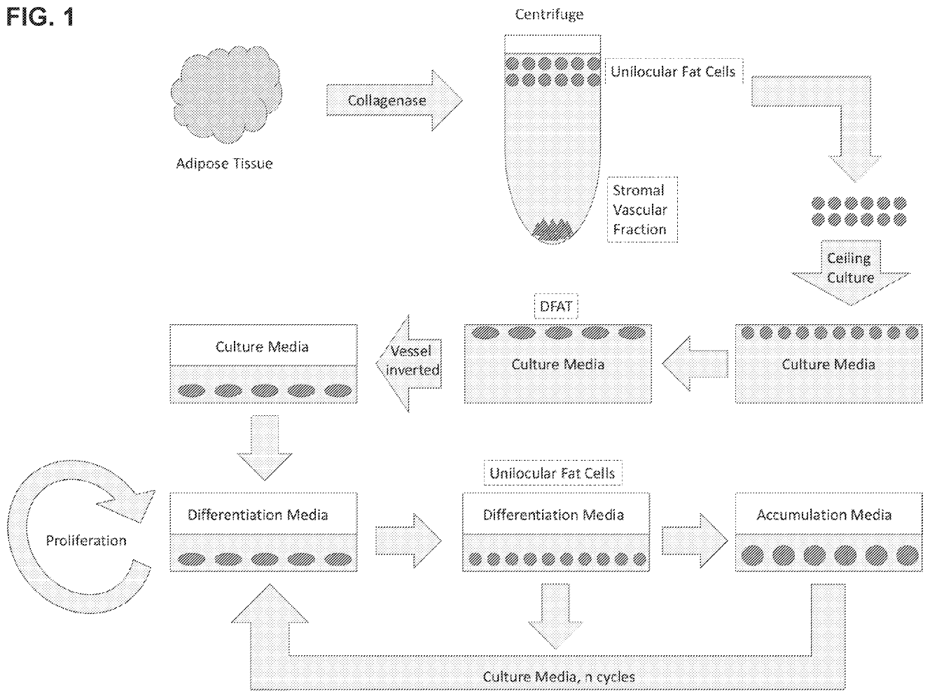

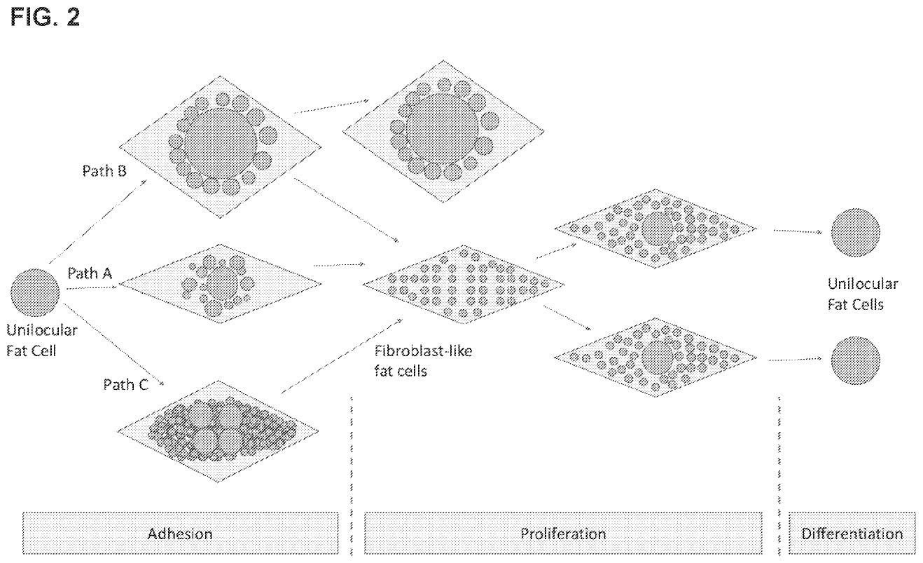

In particular embodiments, mature, isolated adipocytes are dedifferentiated in vitro into DFAT cells. In various embodiments, the mature adipocytes can be isolated in a basal, hypoxic cell culture environment (see FIG. 1). As shown in FIG. 2, when so isolated, the mature adipocytes can expel their accumulated tri-acetyl glyceride (TAG) and adopt a DFAT phenotype, e.g., adopt a fibroblastic morphology.

FIG. 1 represents a simplified dedifferentiation procedural protocol generated by Sugihara et al. (1986), with the addition of the cycling methodology disclosed herein. Embodiments of the present disclosure include methods, systems, and platforms for the isolation of multipotent cells from various species that are capable of differentiation into fat-producing cells. In particular embodiments, the species are avian species.

In some embodiments, adipose tissue is biopsied from a subject. In some embodiments, the subject is a mammalian or avian species. In some embodiments, the avian species is a chicken. Adipocytes can be isolated from the biopsied tissue using standard methods known in the art.

In one embodiment, isolation occurs by mincing and enclosing in a vessel containing a digestion buffer. Briefly, fat tissue (such as a tissue explant) obtained from a subject can be minced (such as through homogenization or by means of other mechanical or chemical techniques) and digested in a digestion buffer solution. In some embodiments, digestion buffer solution is at a minimum a neutral saline solution, which can contain but limited to anti-microbials, growth factors, glucose. In one embodiment, the mass to volume ratio of the tissue to digestion buffer is about 3 to 1. In some embodiments, the mass to volume ratio is less than 3 to 1. In some embodiments, the mass to volume ratio is greater than 3 to 1.

During the adipocyte isolation process, an incubation period can be included in which the digestion buffer and minced tissue are incubated for sufficient time to achieve homogeneous digestion, as reflected by a homogenous, smooth consistency (e.g., without lumps). The tissue and buffer may be agitated during the incubation period (e.g., on a rotating tube rack). In some embodiments, the digestion buffer and minced tissue can be incubated at a temperature above ambient room temperature, for example, at about 37° C., in order to increase the rate of digestion. When the tissue+digestion buffer solution is homogenous, the tissue+digestion buffer solution can be centrifuged until observance of a floating adipose layer (FAL) comprising unilocular fat cells on top of the digestion buffer. In some embodiments, the time required to achieve a homogenous tissue+digestion buffer solution is about 30-45 minutes (e.g., about 30 minutes). One skilled in the art will appreciate that incubation time may be modified by the ratio of enzyme to tissue mass being digested as well as the temperature at which the digestion is conducted. In some embodiments, the specific time of digestion is proportional to the tissue weight and can be modulated by the temperature or other environmental factors such as osmolarity or pH. In some embodiments, the digestion is allowed to continue until a homogeneous solution is observed. Centrifugation at a low rotational speed (e.g., 1500-2000 RPM) can be used to separate isolated adipocytes from the remaining stromal vascular fraction. In some embodiments, centrifugation results in a floating adipocyte layer above the buffer and the stromal vascular fraction (SVF, pelleted) in the container. The tissue+digestion buffer solution can be centrifuged (e.g., approx. 300×g) for a few minutes (e.g., about 5 min). In particular embodiments, centrifugation is performed without filtration. Following centrifugation, the digestion buffer solution and pelleted SVF from below the FAL can then be removed. In some embodiments, at least 90% of the digestion buffer solution and pelleted SVF are removed from the container. For example, at least 90%, 91%, 92%, 93%, 94%, 95%, 96%, 97%, 98%, or 99% of the digestion buffer solution and pelleted SVF are removed from the container. In certain embodiments, the FAL is resuspended in a cell culture medium, centrifuged as described above at least one additional time to achieve the FAL, and the media and any remaining pelleted tissue below the FAL are removed. In some embodiments, at least 90% of the cell culture medium used for washing the FAL is removed from the container. For example, at least 90%, 91%, 92%, 93%, 94%, 95%, 96%, 97%, 98%, or 99% of the cell culture medium is removed from the container. The remaining, isolated adipocytes can be washed several times (e.g., 2, 3, 4, or more times) with cell culture media. In certain embodiments, removal of the digestion buffer and the SVF (and/or the cell culture medium used for washing) is accomplished using a hypodermic needle and syringe.

The isolated mature adipocytes can then be exposed to dedifferentiation conditions. In one embodiment, exposure to dedifferentiation conditions comprises removing the isolated mature adipocytes and transferring the cells to a dedifferentiation container (i.e. a cell culture container such as a culture flask). In certain embodiments, cell culture medium is added to the dedifferentiation container. For example, in some embodiments the FAL is re-suspended in cell culture medium, wherein the flasks or containers containing the fat cells from the FAL are completely filled to the brim with the cell culture medium to create a hypoxic environment. By way of example, in particular embodiments, after placing the isolated mature adipocytes in the dedifferentiation container, the container is filled with cell culture media such that the entire volume of the dedifferentiation container is filled with the isolated mature adipocytes and cell culture media. In some instances, the culturing is performed under hypoxic conditions. For example, the container can then be sealed to prevent gas exchange, e.g., by placing a solid cap or lid on the dedifferentiation container.

The dedifferentiation container can then be incubated for a period sufficient to accumulate a desired population of dedifferentiated fat cells on the ceiling of the container. The incubation may be performed at a standard temperature for culturing animal cells, e.g., about 37° C. In certain embodiments, the dedifferentiation container (culture flask) can be inverted (turned upside down) after being filled and sealed and incubated for a suitable dedifferentiation period to permit certain cells to float through the media and adhere to the top surface of the container or flask (also referred to as the “ceiling” or “ceiling surface”), thereby forming a ceiling culture of DFAT cells. In some embodiments, the dedifferentiation container is undisturbed during the incubation period, thereby promoting adherence of the cells to the ceiling.

In some embodiments, the step of dedifferentiation occurs in the inverted state. In some embodiments, dedifferentiation occurs when there is a solid substrate in contact with mature adipocytes. In some embodiments, during step of dedifferentiation, the adipocytes are co-cultured adipocytes with non-adipocyte cells, such as, for example, one or more of endothelial, epithelial or fibroblast cells. In some embodiments, the non-adipocyte cells are highly proliferative and immortalized. Exemplary non-adipocyte cell lines include, but are not limited to, HEK293, HEK293T, CHO, HELA, 3T3, Jurkat, and Vero.

Dedifferentiation can occur for a period of between 1 day and about 30 days. In some embodiments, the incubation period can last for up to about three weeks. In some embodiments, the period of dedifferentiation is between about 5 and 15 days. The period of dedifferentiation can be about 5, about 6, about 7, about 8, about 9, about 10, about 11, about 12, about 13, about 14, about 15 about 16, about 17, about 18, about 19, about 20 days, or about 21 days. In some embodiments, the incubation period ranges from about three days to about three weeks, three days to about two weeks, three days to one week, five days to about three weeks, five days to about two weeks, five days to one week, one week to three weeks, or one week to two weeks, or any intermediate periods of times within any of these ranges. In some embodiments, the incubation period is about five days to about two weeks.

In some embodiments, dedifferentiation is performed with a serum-containing medium (e.g., fetal bovine serum or fetal calf serum-containing medium). In other embodiments, a dedifferentiation and/or growth medium is used that is free from animal products (e.g., does not contain serum). The growth medium or dedifferentiation medium can be serum-free, e.g., free from FBS or fetal calf serum. In certain embodiments, the dedifferentiation media comprises additives that can encourage dedifferentiation of one or more adipose cells. In some such embodiments, the additives comprise oxygen-scavenging molecules. In some embodiments, the additives comprise citric acid, glucose oxidase, catalase, or a combination thereof. An exemplary cell culture medium can comprise equal volumes of Ham F12 and Dulbecco's modified Eagle's medium supplemented with 10% fetal bovine serum (FBS) or fetal calf serum (FCS) (pH of about 7.2-7.4). The growth media can further comprise additional agents, such as an antibiotic composition, an anti-fungal composition, or a combination thereof. In some embodiments, the growth media can further comprise one or more antibiotics or antimicrobial agents (e.g., about 1% penicillin-streptomycin, about 1% amphotericin b, 1% AntiAnti) or combinations thereof.

In particular embodiments of the present disclosure, de-differentiation is obtained of one or more mature adipocyte cells into DFAT cells. In certain embodiments, the one or more mature adipocyte cells comprise cells obtained from avian species. In particular embodiments, the one or more mature adipocytes comprises chicken adipocytes.

In some embodiments, adipose cells are added to the digestion buffer at a concentration of about 10 grams of adipose cells or tissue explant per about 1 ml of digestion buffer. In some embodiments, the concentration of adipose cells to digestion buffer is about 5 g of cells to about 2 ml of digestion buffer. In some embodiments, the concentration of adipose cells to digestion buffer is about 5 g of cells to about 1 ml of digestion buffer. In some embodiments, the concentration of adipose cells to digestion buffer is about 1 g to about 3 ml. The concentration can be about 1 g of adipose cells to about 1 ml, about 1.5 ml, about 2 ml, about 2.5 ml, about 3 ml, about 3.5 ml, about 4 ml, about 4.5 ml, about 5 ml, about 5.5 ml, about 6 ml, about 6.5 ml, about 7 ml, about 7.5 ml, about 8 ml, about 8.5 ml, about 9 ml, or about 10 ml of digestion buffer. One skilled in the art will appreciate that incubation time may be modified by the ratio of enzyme to tissue mass being digested as well as the temperature at which the digestion is conducted.

### Collagenase

In some embodiments, the digestion buffer comprises a mixture of enzymes. In particular embodiments, the digestion buffer comprises collagenase. The collagenase used in various embodiments can comprise a collagenase that was not derived from an animal. In particular embodiments, the collagenase in the digestion buffer is derived from microbial fermentation.

In some embodiments, the collagenase is an engineered collagenase. In some embodiments, the concentration of collagenase within the digestion buffer can be up to 5% of the total volume of digestion buffer. In embodiments the collagenase concentration is as low as 0.01% of the total volume of digestion buffer. The collagenase concentration can be up to about 0.5%, about 1%, about 1.5%, about 2%, about 2.5%, or about 3%. The concentration of collagenase can be between about 0.1% and 0.3% of the total volume of digestion buffer. In some embodiments, the concentration of collagenase is about 0.2%. The concentration of collagenase can be about 0.10%, about 0.11%, about 0.12%, about 0.13%, about 0.14%, about 0.15%, 0.16%, 0.17%, 0.18%, 0.19%, 0.20%, 0.21%, 0.22%, 0.23%, 0.24%, or 0.25% of the total digestion buffer. In some embodiments, the concentration of collagenase is inversely proportional to the time of digestion, i.e., a lower concentration of collagenase requires a longer time of digestion, and a higher concentration of collagenase decreases the time of digestion.

In some embodiments, the adipose cells are mammalian cells and the collagenase can comprise collagenase type I, type II, or type IV.

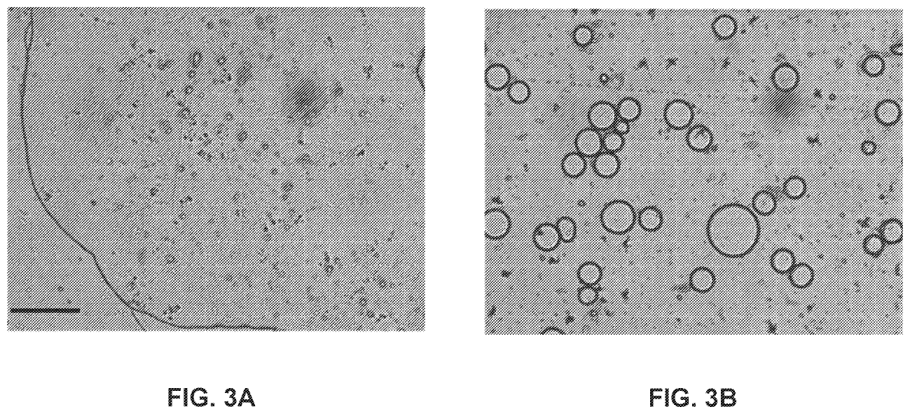

In other embodiments, the adipose cells are avian cells and the collagenase can be collagenase IV as shown, for example, in FIGS. 3A-3B. In some embodiments, the collagenase is a collagenase with a level of tryptic activity that is similar to collagenase IV and less than that of wild-type collagenase I or II. For example, variants of collagenase I or II that contain mutations reducing tryptic activity to a level similar to that of collagenase IV may be used.

### Culturing DFAT Cells

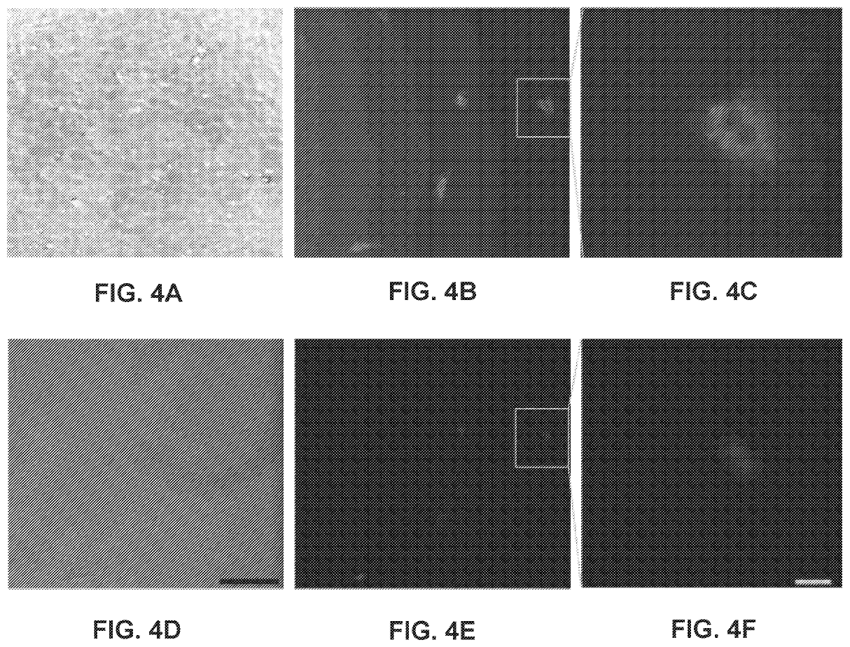

DFAT cells, such as those generated according to the methods disclosed herein, can be removed from the ceiling of the dedifferentiation container by disrupting the adhesion of the cellular body to the container surface. This can be performed by tryptic digestion, scraping, or other routine methods. In certain embodiments, after removal from the container, the suspended cells are filtered through a strainer to remove unwanted particulate matter remaining from the tissue digestion. In some embodiments, the strainer can have a pore size of about 10 micrometers to 1 millimeter, such as approx. 50-500 micrometers, or 50-100 micrometers. Cells can then be propagated according to standard practices of cell culture. In certain embodiments, such standard practices of cell culture include two-dimensional adhesive culture, three-dimensional culture within a hydrogel, various methods of floating culture, or any combination thereof. Cells can then be passaged to a first target biomass. In some embodiments, the first target biomass is equal to a desired level of confluency. In one example, cultured DFAT cells are produced and cultured as shown in FIGS. 4A-4F.

In certain embodiments, DFAT cells are grown in growth media until a first target biomass is attained. In some embodiments, the target biomass is up to about 90% confluence of DFAT cells. In some embodiments, the target biomass is up to about 99% confluence of DFAT cells. In some embodiments, the target biomass is at least about 20% confluence of DFAT cells. The target biomass can be at least about 30% DFAT cell confluence. In certain embodiments, the target biomass is achieved at a DFAT cell confluence of about 30%, about 35%, about 40%, about 45%, about 50%, about 55%, about 60%, about 65%, about 70%, about 75%, about 80%, or about 90%. In one embodiment, the first target biomass is reached upon achieving at least 70% confluence of DFAT cells. In some embodiments, the first target biomass is reached upon achieving at least about 50%, 60%, 65%, 75%, 80%, or more confluence.

### Distinct Populations of DFAT Cells

In certain embodiments, distinct populations of DFAT cells can be generated using the methods disclosed herein, i.e., a plurality of populations of cells (e.g., cell lines) with distinct properties, including multiple distinct populations of DFAT cells originating from a single tissue explant that originated from a single subject or multiple subjects. In some embodiments, multiple, distinct populations of DFAT cells having different properties can be derived from a single population of mature adipocytes from a single subject. In some embodiments, multiple, distinct populations of DFAT cells with different properties can be derived from a homogenous suspension of mature adipocytes.

The distinct populations of DFAT cells can show differences in any of a number of properties, including, but not limited to, growth rate, cell differentiation, conversion of DFAT into fat cells, and/or various morphologic features. For example, in some embodiments the distinct populations comprise cell lines with rapid cell proliferation, while in others the populations comprise fat cells with diverse morphological features. Exemplary properties that can show potential differences between distinct populations include, e.g., doubling time, morphologic features (such as the quantity and quality of lipid beads, distribution of cell diameters, and pseudopodia), telomerase level/activity, viability in culture, ability to re-differentiate, ability and rate of lipid production following re-differentiation, and similarly propensities to differentiate into non-fat cell types (i.e. pericytes, osteoblasts, cardiac muscle cells, etc.), number of insulin receptors, the number silenced genes, epigenetic changes, and others.

Systems and methods for creating or providing multiple distinct populations of DFAT cells with differing properties, e.g., one or more cell lines that can be used in a variety of applications, are further disclosed herein. As such, an aspect of the present disclosure provides isolating or generating at least two cell lines obtained from DFAT cells derived from a single population of mature adipocytes, wherein the at least two cell lines exhibit phenotypic, morphological, genetic, or epigenetic differences relative to one another. In some embodiments, the different cell populations are created using any of the various de-differentiation methods or protocols described herein.

In some embodiments, the distinct populations of DFAT cells are generated by creating a plurality of cell lines derived from DFAT cells obtained from the same source and assessing one or more properties of the cell lines to identify those displaying a phenotypic profile of interest. Without being bound by the following theory, it is believed that the differences observed in the distinct cell lines reflect heritable variations existing within populations of DFAT cells generated using the present methods (e.g., reflecting genetic mutations or epigenetic changes occurring by chance during dedifferentiation or prior to tissue extraction, leading to genetic or epigenetic differences between the individual cells used to establish the distinct cell lines).

In some embodiments, distinct populations of DFAT cells obtained from the same source (e.g., cell lines originating from the same subject and/or from the same suspension of mature adipocytes) but displaying different properties are isolated under different conditions, e.g., by following dedifferentiation protocols differing in one or more aspect or condition (e.g., differing with respect to one or more reagents, reagent concentrations, temperature, container, etc.). In other embodiments, distinct populations of DFAT cells obtained from the same source (e.g., cell lines originating from the same subject and/or from the same suspension of mature adipocytes) but displaying different properties are generated using the same dedifferentiation protocol, but are isolated at a different time point in the protocol (e.g., following a different number of hours or days of dedifferentiation).

The multiple distinct cell populations generated using the methods disclosed herein can be confirmed and characterized in any manner known by one of skill in the art, e.g., via epigenetic analysis, RNA sequencing, microscopy, or other methods of characterizing and phenotyping populations of cells. In particular embodiments, the distinct populations are derived from avian cells, e.g., chicken cells, turkey cells, duck cells, pigeon cells, or others. In other embodiments, the multiple distinct cell populations are derived from mammalian cells.

Also disclosed herein are methods of creating at least two distinct populations of mature adipocytes, wherein the different populations are derived from re-differentiated mature adipocytes as described herein.

### Re-Differentiation of DFAT Cells

In some embodiments, one or more subculture DFAT populations created according the methods described herein are re-differentiated into mature adipocytes. Generally, re-differentiation of the one or more subculture populations of DFAT cells comprises contacting the cells with a differentiation medium that comprises one or more re-differentiation inducers. Such a differentiation medium can be used to induce changes to the morphology of the dedifferentiated cell. In particular embodiments, a differentiation medium induces the differentiation of the DFAT cells into another cell type. For example, as described herein, the introduction of a differentiation medium into the cellular environment can induce a process of lipid production and accumulation within the cells. Cells that have been seeded at a density appropriate for such induction into lipid-producing cells can be exposed to a differentiation medium for a time period that is sufficient to result in the development of a morphology and lipid content that is similar to that of mature adipocytes.

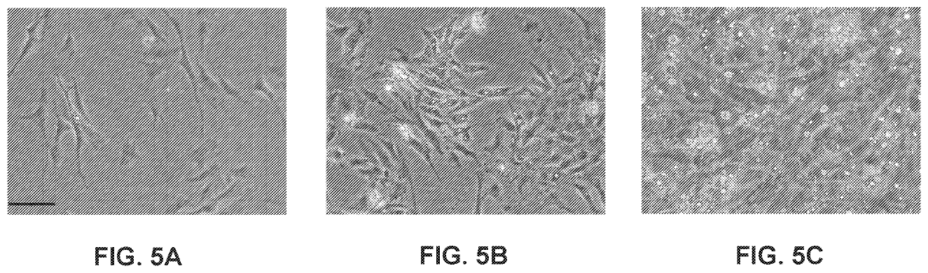

Re-differentiation of the adipose cells can be detected in any of a number of was. For example, re-differentiation can be detected via a notable increase in the accumulation of lipid droplets, and/or by the development of adipocyte morphology by the cells, e.g., a more rounded morphology. In some embodiments, re-differentiation can be assessed as described in FIGS. 5A-5C.

In exemplary embodiments, the differentiation medium comprises at least one redifferentiation inducer. The at least one re-differentiation inducer can be configured to re-differentiate DFAT cells into adipocytes that comprise triacylglycerol (TAG) vesicles that are morphologically similar to those of naturally occurring mature adipocytes.

In particular embodiments, the redifferentiation inducer is insulin. In some embodiments, the redifferentiation media comprises any one or more of insulin, 3-isobutyl-1-methylxanthine, insulin-transferrin-selenium-X supplement, indomethacin, dexamethasone, rosiglitazone, biotin, pantothenate, a PPARγ agonist, sodium oleate, or wheat germ agglutinin. For example, in various embodiments, the redifferentiation media can comprise about 1-10 μg/ml insulin, about 0.45-0.55 mM 3-isobutyl-1-methylxanthine, about 0.2-1 μM insulin-transferrin-selenium-X supplement, about 100-500 μM indomethacin, about 0.25 μM-1 mM dexamethasone, a millimolar concentration of rosiglitazone, about 10-50 M of biotin, about 10-30 mM pantothenate, a millimolar concentration of at least one PPARγ agonist, about 100-200 μM sodium oleate, a millimolar concentration of wheat germ agglutinin, or any combination of any one or more of these redifferentiation inducers at any of the indicated exemplary concentrations.

In some embodiments, re-differentiation is induced using a re-differentiation coating. For example, re-differentiation can comprise manipulating a mechanical property of the culture environment. In some embodiments, carrier particles are added to the culture environment. In some embodiments, any of one or more features such as growth media density, growth media osmolarity, and growth media temperature can be manipulated to encourage re-differentiation of DFAT cells. In some embodiments, certain factors can be added to encourage redifferentiation. One such exemplary factor is a mitogen. In some embodiments, agents are introduced during re-differentiation that are known to control or limit the formation of non-adipocyte cells. One example of such an agent is trypsin.

### DFAT and Redifferentiation Cycling

The present disclosure is also based in part on the surprising discovery that DFAT cells produced using the herein-described methods can be cycled, e.g., taken through repeated rounds of dedifferentiation and re-differentiation. For example, in particular embodiments, cells are repeatedly cycled between the proliferative DFAT state and the mature adipocyte phenotype. Cells can be cyclically switched between these states without the aid of direct genetic manipulation.

By way of example, in particular embodiments mature adipocytes generated as described herein are dedifferentiated to regenerate DFAT cells. In some embodiments, the process for dedifferentiation of the generated mature adipocytes comprises any of the dedifferentiation methods described in the present disclosure. A subpopulation of the resulting DFAT cells (or the “resultant DFAT cells”) can then be re-differentiated, e.g., using any of the redifferentiation methods described herein. As such, in certain embodiments, repeated rounds of alternating dedifferentiation and redifferentiation of the cells amounts to a method for cycling the cells, e.g., cycling with respect to their differentiation state, morphology, and function.

In some embodiments, following the initial dedifferentiation of isolated mature adipocytes and their subsequent re-differentiation as described above, the step of dedifferentiating in each subsequent cycle is performed on a population of the re-differentiated mature adipocytes that were re-differentiated in the immediately preceding cycle.

In some embodiments, the cycling process can comprise the steps of culturing, subculturing, or a combination thereof during each cycle. For instance, a population of DFAT cells can be created in accordance with any of the methods described herein, and then cultured to reach a target biomass. The DFAT cells within the target biomass can then be re-differentiated into mature adipocytes. Alternatively, the DFAT cells within the target biomass can be subcultured into one or more subculture populations of DFAT cells, wherein at least one of the subculture populations of DAFT cells is re-differentiated into mature adipocytes, and at least one of the subculture populations of DFAT cells is permitted to proliferate to a second target biomass.

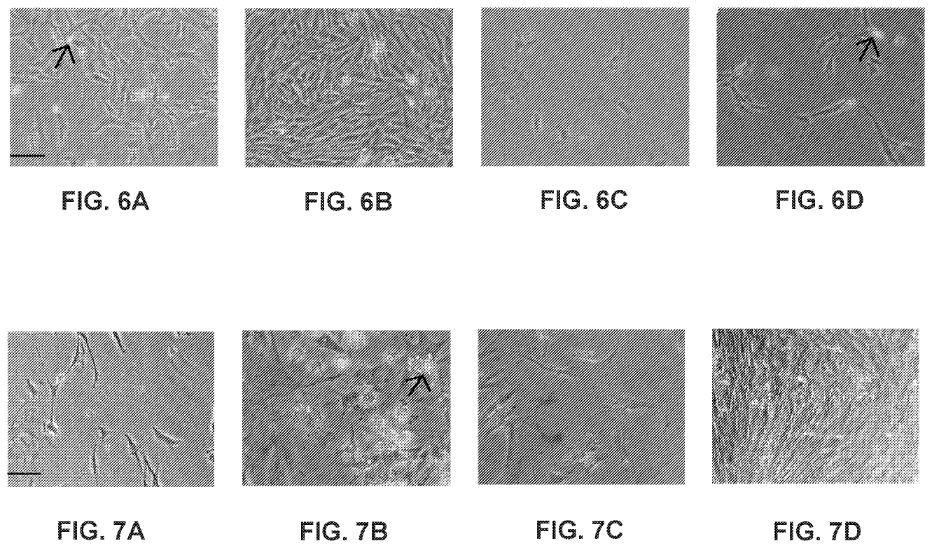

For example, in one embodiment, DFAT cells are induced to differentiate by introducing differentiation media to the cellular environment. Differentiation of the DFAT cells into adipocytes can be detected, e.g., by observing large lipid droplets within one or more of the re-differentiated cells. The differentiation medium can then be removed, leading to a loss of lipid accumulation among the cells and the adoption of a more elongated morphology characteristic of pluripotent cells. The cells can then be passaged, and differentiation media reintroduced, leading once again to lipid accumulation in the cells. In some embodiments, DFAT differentiation can be performed as described for FIGS. 6A-6D and/or FIGS. 7A-7D.

In some embodiments, the re-differentiated mature adipocytes can be divided into two portions, e.g., a first portion and a second portion. For example, the first portion can comprise a de-differentiation population, and the second portion can comprise a harvest population. In some embodiments, the dedifferentiation population is subjected to any of the various dedifferentiation methods described herein, and the harvest population is collected as resultant fat-producing cells. For example, the harvest population can be collected as a lipid-containing or lipid-producing product for use in any of the variety of applications disclosed herein. In some embodiments, the harvest population is separated from the dedifferentiation population by permitting cells of the harvest population to accumulate lipids and float in a suspension environment. In some such embodiments, the harvest population comprises one or more cells that float in a suspension. In one aspect, the present disclosure provides a harvest population of lipid-producing cells derived from DFAT cells as described herein. In some embodiments, the dedifferentiation population comprises one or more adipocytes that do not float in a suspension.

The cycling of DFAT cells as described herein can be performed for a given number of cycles, or can be performed indefinitely. In some embodiments, the cycling occurs without a pre-defined end-point, but rather continues until the viability of the resultant cells is substantially decreased. In certain embodiments, the cells undergo at least two cycles of creating DFAT cells from previously re-differentiated mature adipocytes. In some embodiments, the cells undergo up to 100 cycles. By way of non-limiting example, cycling can be performed for at least 1, 2, 3, 4, 5, 10, 15, 20, 25, 30, 35, 40, 45, 50, or 100 cycles. Such cycling can be performed up to 50 times, up to 100 times, up to 200 times, up to 300 times, up to 400 times, up to 500 times, up to 600 times, up to 1,000 times, up to 10,000 times, or up to 50,000 times. In some embodiments, such cycling can be performed for an indefinite number of cycles such that an immortal cell line is created. In some embodiments, the cycling is performed until the re-differentiated mature adipocytes show a detectable decrease (e.g., decrease of 5%, 10%, 15%, 20%, 30%, 40%, or more) in their ability to dedifferentiate and then proliferate, and/or to synthesize and store fat. In some embodiments, the number of cycle repetitions can vary based upon the particular primary DFAT cells that are isolated. For example, the number of cycle repetitions can vary based on the site from which the mature adipocytes were obtained, the age of the subject, the species or genus of the subject, the health status of the subject, or a combination thereof. In some embodiments, the number of cycles can vary between different distinct DFAT cell populations as described elsewhere herein.

As described in various embodiments disclosed herein, cycling can be promoted using various agents such as mitogens and/or differentiation factors. Cycling of DFAT cells using the present methods can be used, e.g., to provide a source of lab-grown fat cells such as the resultant fat-producing cell lines disclosed herein.

Generation of DFAT Cells from Various Species.

The methods described herein can be utilized with subjects of any species. For example, the present methods can be applied to cells obtained from any species that comprises adipocytes or adipose tissue. For example, the method can be applied to cells from mammals, birds, fish, reptiles, or any combination thereof. Isolated cells or adipocytes can comprise sheep cells, goat cells, horse cells, cow cells, human cells, monkey cells, mouse cells, rat cells, rabbit cells, canine cells, feline cells, porcine cells, chicken cells, turkey cells, duck cells, pigeon cells, guinea fowl cells, ostrich cells, or cells from other non-human animals.

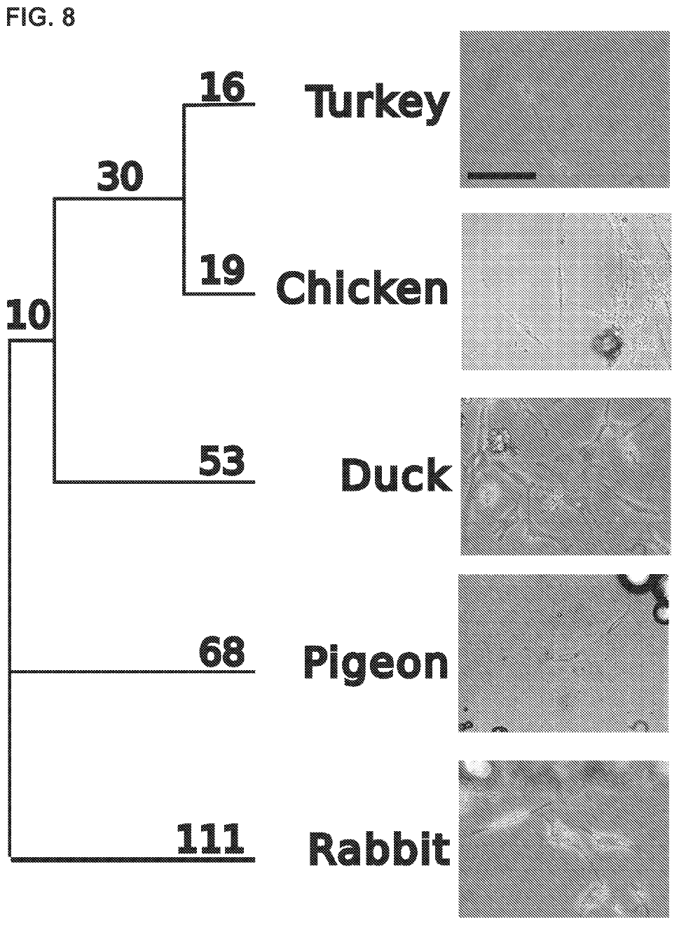

By way of example, FIG. 8 provides images of DFAT cells derived from various species as observed on the ceilings of de-differentiation containers when following the herein disclosed methods. The species are arranged on a cladogram according to their molecular relationships, to demonstrate the substantial evolutionary space within which the present methods can be applied. The evolutionary relationships are reported in the length of each branch segment in millions of years (MY). The specific values were computed by aligning published 18s rRNA sequences and applying a conservative molecular clock coefficient to adjust the units. From top to bottom, the pictorial micrographs show DFAT cells derived from turkey, chicken, duck, pigeon, and rabbit. This broad evolutionary cross section illustrates that the present methods can be applied to all branches of avian species routinely raised for human consumption, as well as to mammals. The methods can also be used in other adipose-containing vertebrates, including reptiles, amphibians, and fish.

### Fat-Producing Cell Lines

In some embodiments of the present disclosure, fat-producing cell lines are provided that can survive or grow in vitro under conditions appropriate for human consumption, as are methods of making the same. Such cell lines can be created according to any one or more of the various method embodiments disclosed herein. Such fat-producing cell lines can be referred to herein as “resultant fat-producing cell line,” “resultant cell line,” “resultant cells,” “resultant mature adipocytes,” and the like. The present disclosure provides fat-producing, cells, cell lines, populations or pluralities of cells produced according to any one or more of the present methods, e.g., cells or cell lines generated or maintained using any of the cyclical culture methods disclosed herein. The present disclosure also provides methods of making and using the cells and cell lines, e.g., for the production or enhancement of an alternative meat product.

In some embodiments, the fat-producing cell lines described herein can grow in vitro under food safe conditions. Certain fat-producing cell lines created according to the present methods maintain the ability for cyclical dedifferentiation, proliferation, and re-differentiation for an extended period of time. The present fat-producing cell lines can be configured, e.g., to synthesize and store fat for an extended period of time. In some embodiments, the extended period of time comprises at least one week. In some embodiments, the extend period of time comprises at least one year. In some embodiments, the extended period of time comprises at least about one month, about two months, about three months, about four months, about five months, about six months, about seven months, about eight months, about nine months, about ten months, about eleven months, or about twelve months. In some embodiments the resultant fat-producing cell lines are immortal.

In some embodiments, the present fat-producing cell lines, or any population of DFAT cells derived using the methods disclosed herein, can exhibit a population doubling over a 24- to 48-hour passage period with <10% senescence over a period of at least six months, at least 12 months, at least 18 months, or at least 24 months. In some embodiments, the resultant fat-producing cell lines comprise inducible cycling of pluripotency over at least a four-month period. The present fat-producing cell lines can show an inducible lipid accumulation of, e.g., at least 50%, 55%, 60%, 65%, 70%, 75%, 80%, 85%, 90%, 95%, or more of the intracellular volume over at least a four-month period, at least a six month period, at least a 12 month period, at least an 18 month period, or at least a 24 month period. In some embodiments, the fat-producing cell lines substantially retain lipid production ability over time when cycled as described in this disclosure. In some embodiments, a fat-producing cell line can retain the ability to produce a total lipid accumulation of at least 50%, 55%, 60%, 65%, 70%, 75%, 80%, 85%, 90%, 95% of an intracellular volume after 6 months, 12 months, or 18 months of culturing as compared to the total lipid accumulation produced by the fat-producing cell line at its first passage. In one example, a fat-producing cell line that produces an internal volume of lipid accumulation on average of 90% at its first passage may produce an of lipid accumulation on average of 81% after 12 months of continuous cycling. In some embodiments, the present fat-producing cell lines can survive a suspension culture for at least 13 passages (or, e.g., at least 10, 11, 12, 14, 15, 16, 17, 18, 19, 20, or more passages). In certain embodiments, the present fat-producing cell lines are adaptable to commercial scale bioreactors.

In some embodiments, inducible cycling of pluripotency is assessed via indices such as telomerase activity and/or the presence of canonical stem-cell markers. In some such embodiments, telomerase activity is measured via the standard telomeric repeat amplification protocol (TRAP. Canonical stem cell markers can be evaluated, e.g., in bulk RNA-seq experiments or using any other pertinent method of measuring enzymatic activity.

The extent of inducible lipid accumulation in resultant fat-producing cells can be used as a direct, representative measure of the commercially valuable animal fat that can be generated from the cells. In some embodiments, inducible lipid accumulation is measured using fluorescence microscopy. In some such embodiments, lipids can be visually distinguished by one skilled in the art, or can be stained with, e.g., triacylglycerol (TAG)-targeting Nile Red dye for visualization and quantification, or can be quantified using any other pertinent method for measuring lipid content.

In some embodiments, the resultant fat-producing cell line comprises a commercially scalable cell line as understood by one skilled in the art.

### Uses of the Re-Differentiated Adipose Cells (i.e., Resultant Fat-Producing Cells)

Further aspects of the present invention include methods of using one or more of the re-differentiated adipose cells created through any of the various methods disclosed herein. In certain embodiments, non-limiting, exemplary uses of the re-differentiated adipose cells comprise use in perfume, meat alternatives, cosmetics, soaps, moisturizers, wood working or furnisher finishes, flavor-discovery platforms, commercial lubricants, birdseed feeders, fuel, or any combination thereof. Thus, an aspect of this disclosure are such products comprising re-differentiated adipose cells made according to the herein-described methods.

### 1. Cosmetics

In certain embodiments, the present cyclical adipocyte production methods and compositions can be used in cosmetics where lipids are used. In some embodiments, cells produced according to the present methods can be used in lipofilling injections for regenerative medicine purposes. Such embodiments can be utilized, e.g., for cosmetic surgery, including for lipofilling to create additional volume in certain body parts of a subject or patient. By way of non-limiting example, cells generated according to the present disclosure can be utilized for lipofilling of a subject's face, hands, neck, breasts, buttocks, arms, legs, feet, or any combination thereof.

In some such embodiments, in a clinical setting a biopsy can be obtained, wherein the biopsy comprises adipocyte tissue from a subject and, using the herein-disclosed methods, the adipocyte tissue can be used to cyclically produce DFAT cells or one or more populations of mature adipocytes, e.g., autologous DFAT cells or mature adipocytes, for use in a lipofilling injection. In some embodiments, the DFAT cells and/or mature adipocytes produced according to the present methods are kept, e.g., in cold storage. As used herein, cold storage indicates any temperature below freezing, e.g., using a storage container or freezer that is at least as cool as about −20 degrees C., or at least as cool as about −80 degrees C.

In some embodiments, the mature adipocytes, the DFAT cells, or both that are produced according to the presently disclosed embodiments can be cyclically produced such that another biopsy is not required in the future. As such, the. Present methods can be used to reduce the amount of primary tissue needed to provide treatment in perpetuity through the use of a single biopsy.

In particular embodiments, the injections are: (1) produced by blending one or more primary tissues or cell types such as adipocyte tissue, connective tissue, SVF, or ADSCs, with the cyclically produced DFAT cells or the resultant mature adipocytes, or (2) produced entirely by culturing DFAT cells or the resultant mature adipocytes. In any such embodiments, these components can be combined with other standard ancillary components, such as saline solution to maintain equilibrium at the injection site.

In some embodiments, cells produced according to the methods described herein can be utilized for formulas or preparations that assist with or promote skin moisturization, skin hydration, or a combination thereof. Exemplary formulations include, but are not limited to moisturizers, creams, and lotions.

In some embodiments, cells produced according to the present methods can be used in emollients, which help make the skin soft and smooth by filling in gaps between skin cells. By way of non-limiting example, such products can include creams, lotions, and lip balms.

In various embodiments, cells produced according to the methods described herein can be utilized for methods and products related to, e.g., skin repair and protection, skin cleansing and makeup removal, soaps, hair care, lip care, sunscreen, fragrance delivery, or any combinations thereof. In certain embodiments, cells produced according to the methods described herein can be used in cosmetic formulations to enhance or improve the texture or consistency of products such as creams and lotions. In some embodiments, cells produced in accordance with the methods described herein can be utilized in formulations to improve the stability or shelf life of cosmetic products, such as by acting as antioxidants or preservatives, preventing the oxidation and degradation of other ingredients.

In some embodiments, the present methods and compositions can be used in the context of cosmetic surgery, where ADSCs and adipocytes are used interchangeably with cyclically produced DFAT and adipocytes. For instance, the cells produced according to the present disclosure can be used in tissue regeneration and/or volume enhancement. Without being bound by the following theory, it is believed that DFAT cells created according to the present disclosure exhibit regenerative properties and can promote tissue repair and regeneration. As such, when injected into the target area, it is believed that such cells can, e.g., enhance blood supply, improve tissue quality, and/or stimulate the growth of new fat cells. In addition, cells produced according to the present methods can be used in fat grafting procedures, including breast augmentation, facial rejuvenation, buttocks augmentation, hand rejuvenation, for the correction of deformities or irregularities resulting from previous surgeries or trauma, or any combination thereof.

A particular advantage of utilizing autologous DFAT cells derived according to the presently disclosed methods is that, when used as the initial adipocyte tissue, there is a reduced risk of allergic reactions, graft rejection, or foreign body reactions compared to synthetic fillers or implants. In some embodiments, however, the present methods can also be performed using allogeneic DFAT cells.

### 2. Perfumery or Fragrance Design

The methods and compositions disclosed herein can also be used for perfumery or fragrance design where lipids are used. By way of non-limiting example, cells produced according to the methods disclosed herein can be utilized in the production of carrier oils that can be used to dilute and extend fragrance oils, solid perfume formulations, encapsulation of fragrance molecules, stability and preservation of fragrance components, or any combination thereof.

### 3. Flavor and Taste Design

In some embodiments, cells produced using the herein-described methods can be utilized in methods and products related to flavor and taste design where lipids are traditionally used.

By way of non-limiting example, cells derived according the presently disclosed methods can be utilized to assist with flavor solubility and dispersion, flavor enhancement (such as by improving the release and transport of aromatic molecules to taste buds), improving the mouthfeel and texture of food products, for flavor protection (such as by preventing or reducing the effects of oxidation or exposure to air and light), encapsulation of flavor compounds, flavor retention (such as through use of the cells in lipid-based marinades or cooking oils), flavor pairing, aroma and flavor development, lipid-based flavor delivery systems, or any combination thereof.

### 4. Lubrication

The methods and compositions disclosed herein can also be utilized in methods and products related to lubrication in, e.g., industrial, equipment, automotive, medical, and pharmaceutical fields where lipids are traditionally used. By way of non-limiting example, cells derived according to the presently disclosed methods can be utilized as base oils and esters, lubricating oils (such as engine oils) to reduce friction and wear between moving parts, as extreme pressure additives to enhance a lubricant's film strength and load-carrying capacity, as lipid-based friction modifiers to reduce friction and improve fuel efficiency in engines, as biodegradable lubricants, for boundary lubrication, as automotive and industrial greases, as biocompatible lubricants for use in medical and pharmaceutical applications, or any combination thereof.

### 5. Drug Discovery in Human and Non-Human Science

In some embodiments, cells produced according to the present methods can be used in methods and products related to drug discovery, e.g., in human and non-human medical and pharmaceutical fields where lipids and adipocytes are traditionally used. By way of non-limiting example, cells produced according to the methods disclosed herein can be utilized in creating lipid targets for pharmaceutical preparations (e.g., cholesterols, fatty acids, sphingolipids, prostaglandins, leukotrienes). In certain embodiments, cells produced according to the methods herein can be utilized for in the analysis of lipid species in biological samples. Cells produced as described herein can also be useful in identifying lipid biomarkers associated with diseases or drug responses, or for understanding lipid metabolism and its role in health and disease.

In some embodiments, cells produced according to the methods disclosed herein can be useful in drug formulation and delivery (e.g. in lipid-based drug delivery systems, lipid nanoparticles, liposomes, lipid-based emulsions to improve a drug's solubility and bioavailability, and lipid-based drug carriers to target specific tissues or cells), drug screening assays (e.g. incorporation of the cells into microfluidic devices to study drug effects on cell membrane behavior, or lipid-based assays to screen compounds for their lipid-binding properties), or the validation of lipid-related drug targets.

In additional embodiments, adipocytes generated using the methods disclosed herein can be utilized in studying drug effects and developing treatments. By way of non-liming example, such resultant adipocytes can be utilized for obesity and metabolic disease research (e.g., the culturing of adipocytes to investigate how they respond to various factors, such as hormones, nutrients, and drug compounds), screening for anti-obesity drugs, in models for the validation of drug targets, to study the inflammatory responses of adipose tissues and for screening drugs that may modulate these responses, in studies on lipid metabolism and lipidomics, for drug formulation and delivery, pharmacological and toxicological studies, and drug safety testing (e.g. researching the potential deleterious effects that a given drug or compound may have on adipocyte function or lipid metabolism).

An additional use of the fat-producing cells derived according to the presently disclosed methods is in personalized medicine. For instance, cells generated from autologous adipocytes can be used in personalized medicine approaches. By way of example, cyclical dedifferentiation and redifferentiation and culturing of resultant adipocytes from individual patients, can permit researchers to test drug responses and design treatment plans tailored to a patient's specific metabolic profile.

### 6. Pet Food Production

In some embodiments, cells produced using the herein-described methods can also be used in methods and products related to food production of pet food where lipids and adipocytes are traditionally used. By way of example, cells derived according to the methods of the present disclosure can be utilized in pet food as an energy source, as essential fatty acids, to improve the palatability and flavor of pet food, to improve texture and mouthfeel, to assist with nutrient absorption, preservatives, or any combination thereof.

7. Blends with Used Cooking Oil

The present methods and compositions can also be used in products and methods related to applications involving leftover or used cooking oil. By way of example, cells produced according to the methods disclosed herein can be blended with a used cooking oil to create a proprietary blend that can be more useful than the used cooking oil alone. Potential products or industries where such an blend can be useful include: biodiesel production, animal feed, industrial and agricultural uses (such as use as an industrial lubricant or as an ingredient in the manufacturing of various industrial products, such as soaps, detergents, paints, and bio-based plastics, a component in agricultural pesticides or herbicides to enhance their performance and effectiveness), composting and soil amendment, energy generation (e.g. as a source of energy through combustion or conversion into biogas), or any combination thereof.

### 8. Biofuels

The present methods and compositions can also be utilized in products and methods related to applications involving biofuel. For instance, cells produced according to the methods disclosed herein can be used in hydrotreated renewable jet fuel (HRJ), which is a sustainable aviation fuel that is traditionally derived from various renewable feedstocks, including, as an example, chicken or pork fat.

The present methods and compositions can also be utilized in products and methods related to applications involving petroleum blends. For instance, cells produced according to the methods disclosed herein can be blended with a fuel such as gasoline as a potential strategy to reduce greenhouse gas emissions and enhance the sustainability of the transportation sector.

### 9. Biologic Pharmaceuticals

In some embodiments, the present methods and compositions can be utilized in products and methods related to biologic pharmaceuticals. For example, cells produced according to the methods disclosed herein can be used in several ways in the development and delivery of such pharmaceuticals. By way of non-limiting examples, cells produced according to the present methods can be used to stabilize and protect biologic drugs, as lipid-based carriers to encapsulate and protect a biologic drug, to improve the oral delivery of biologics by protecting them from degradation in the gastrointestinal tract and facilitating their absorption into the bloodstream, for topical and transdermal delivery of biologics, as adjuvants in certain vaccine formulations to enhance the immune response to biologic antigens, as a source of energy and essential fatty acids for cell culture media, as lipid nanoparticles to deliver genetic material, such as viral vectors carrying therapeutic genes, into target cells, or any combinations thereof.

### 10. Leather Production

In yet another exemplary embodiment, the present methods and compositions can be used in methods and products related the field of leather production. Briefly, and by way of non-limiting embodiments, cells produced according to the methods disclosed herein can be utilized in the tanning process (e.g., interacting with the collagen fibers in an animal hide to making the leather more rigid and less susceptible to decomposition), in the fatliquoring process (e.g. the cells can be applied to the surface of leather to increase the leather's pliability), the finishing process (e.g., adding waxes, oils, and other substances containing the cells to enhance the leather's appearance, feel, and durability), the polishing process (e.g. creams or waxes containing the cells to provide a smooth surface finish), in leather care products (e.g. cleaners, conditions, etc.), or any combinations thereof.

### 11. Regenerative Medicine

In another exemplary embodiment, the present methods and compositions can be used to create a large quantity of DFAT cells which can be used in place of ADSCs for stem-cell injections. Such injections show promise, e.g., by allowing the injected stem cells to differentiate to a terminal state by natural processes in the body, or through the release of mitogenic factors by the DFAT cells that can aid in the treatment of, e.g., age-related disorders and/or cancers. By way of non-limiting example, patient adipose tissue could be biopsied, and the methods disclosed herein used to dedifferentiate and then iteratively cycle the cells until a target biomass is achieved. The target biomass of DFAT cells could then be reinjected into the patient, e.g., using a protocol analogous to current protocols for ADSC injections or other stem-cell injections.

### 12. Growth Media

In some embodiments, one or more of the various media disclosed herein can comprise only food-safe components. As used herein, the phrase “food-safe components” can refer to ingredients that are deemed suitable for human consumption by a governmental agency. “Food-safe components” can refer, for example, to any ingredient that is deemed suitable for consumption by the U.S. Food and Drug Administration or the U.S. Department of Agriculture.

In some embodiments, serum-free cell cultures are prepared, e.g., based on methods described in Messmer et al. (2022), the entire disclosure of which is herein incorporated by reference. In some embodiments, wells are seeded at 10⁵ cells/cm2 cells per well and allowed to adhere under normal FBS-containing media. At time point 0, three wells can be trypsinized and frozen, and the remaining twelve wells can be washed and exposed to FBS-free media. Such embodiments comprise cells that have been starved of animal serum. At 0.5, 1, 2, and 24 hours, sets of three replicates can be washed and frozen. In some embodiments, these cells can then undergo RNA-seq analysis, e.g. and differential gene expression studies performed on the RNA-seq data to identify starvation-related genes. Data from the starved cells can then be compared, e.g., to data from cells that were not exposed to animal-free serum, e.g., to assess the impact of the media used on gene expression/surface protein receptor up- or down-regulation during serum starvation, e.g., for ligant receptors and transporter proteins. Using these differentially expressed genes, the components that the cells are lacking can be predicted. In exemplary embodiments, gene expression indicative of surface receptor upregulation is used to identify corresponding media components most sought after by the cells.

### Exemplary Embodiments

As used below, any reference to a series of embodiments is to be understood as a reference to each of those embodiments disjunctively (e.g., “Embodiments 1-4” is to be understood as “Embodiments 1, 2, 3, or 4”).

Embodiment 1. A method of cyclical production of fat cells, the method comprising: providing a plurality of mature adipocytes; incubating the plurality of mature adipocytes under conditions such that at least a portion of the plurality of mature adipocytes dedifferentiates, thereby generating a plurality of dedifferentiated fat cells (DFAT cells); culturing the plurality of DFAT cells in a growth medium until a first target biomass is obtained; subculturing the first target biomass in one or more subculture populations of DFAT cells; and incubating the one or more subculture populations of DFAT cells under conditions such that at least a portion of the one or more subculture populations re-differentiates into one or more populations of re-differentiated mature adipocytes.

Embodiment 2. The method of embodiment 1, further comprising: dividing at least one of the one or more populations of re-differentiated mature adipocytes into a first and a second subpopulation; incubating the first subpopulation of re-differentiated mature adipocytes under conditions such that at least a portion of the first subpopulation of re-differentiated mature adipocytes dedifferentiates, thereby generating a plurality of second generation DFAT cells; and culturing the second subpopulation of re-differentiated mature adipocytes under conditions such that at least a portion of the second subpopulation of re-differentiated mature adipocytes accumulate a target lipid mass.

Embodiment 3. The method of embodiment 1 or 2, wherein the target lipid mass is about 20-90% of the cell volume.

Embodiment 4. The method of embodiment 2 or 3, wherein the second subpopulation of re-differentiated mature adipocytes is cultured in a culture medium comprising glucose at a concentration of at least 4 g/L, optionally, 4 g/L-15 g/L, or 4 g/L-10 g/L, or 4 g/L-8 g/L.

Embodiment 5. The method of any one of embodiments 1-4, wherein the one or more subculture populations of DFAT cells are cultured in step v) in a re-differentiation medium comprising one or more components selected from the group consisting of insulin, 3-isobutyl-1-methylxanthine, insulin-transferrin-selenium-X supplement, indomethacin, dexamethasone, rosiglitazone, biotin, pantothenate, a PPARγ agonist, sodium oleate, and wheat germ agglutinin.

Embodiment 6. The method of any one or more of embodiments 2-5, further comprising: culturing the second generation of DFAT cells in a growth medium until a second target biomass is obtained; subculturing the second target biomass into a second generation of subcultured DFAT cells; and incubating the second generation of subcultured DFAT cells under conditions such that at least a portion of the second generation of subcultured DFAT cells re-differentiates into a second generation of re-differentiated mature adipocytes.

Embodiment 7. The method of any one or more of embodiments 1-6, further comprising cyclically repeating one or more of steps ii), iii), iv), or v) for a total of n cycles, wherein the step ii) for each cycle is performed on the re-differentiated mature adipocytes that were re-differentiated in step v) of the immediately preceding cycle, wherein n is equal to the number of cycles during which the re-differentiated mature adipocytes maintain the ability to proliferate and/or maintain the ability to synthesize and store fat.

Embodiment 8. The method of any one or more of embodiments 1-7, wherein the plurality of mature adipocytes is isolated from adipose cells or from a tissue explant comprising adipose tissue.

Embodiment 9. The method of embodiment 8, wherein the plurality of mature adipocytes is isolated by: adding the adipose cells or tissue explant to a digestion buffer in a container; incubating the adipose cells or tissue explant in the digestion buffer for at least about 30 minutes; centrifuging the adipose cells and digestion buffer to create a first floating adipocyte layer (FAL) above the digestion buffer and a pelleted tissue at the bottom of the container; removing the digestion buffer and pelleted tissue from the container; suspending the first FAL in growth media; centrifuging the first FAL and growth media to create a second FAL and a second pelleted tissue; removing the digestion buffer and the second pelleted tissue from the container; and suspending the second FAL in growth medium, wherein the second FAL comprises the mature adipocytes.

Embodiment 10. The method of embodiment 9, wherein the adipose cells are added to the digestion buffer at a concentration of about 1 gram of adipose cells or tissue explant per 3 ml of digestion buffer.

Embodiment 11. The method of embodiment 9 or 10, wherein the digestion buffer comprises a collagenase.

Embodiment 12. The method of embodiment 11, wherein the collagenase is derived from microbial fermentation.

Embodiment 13. The method of embodiment 11 or 12, wherein the collagenase is collagenase IV.

Embodiment 14. The method of any one or more of embodiments 9-13, wherein the digestion buffer comprises between 0.2% and 1% collagenase IV.

Embodiment 15. The method of embodiment 13 or 14, wherein the collagenase IV is from Clostridium histolyticum.

Embodiment 16. The method of any one or more of embodiments 8-15, wherein the tissue explant comprises subcutaneous fat extracted from a subject.

Embodiment 17. The method of embodiment 16, wherein the subject is a mammal, a bird, a fish, a reptile, a crustacean, or an amphibian.

Embodiment 18. The method of any one or more of embodiments 8-17, wherein the adipose cells do not comprise or were not derived from pluripotent stem cells, embryonic stem cells, or undifferentiated stem cells.

Embodiment 19. The method of any one or more of embodiments 8-18, wherein the adipose cells comprise primary mammalian cells, primary avian cells, primary fish cells, primary reptilian cells, primary crustacean cells, primary amphibian cells, or a combination thereof.

Embodiment 20. The method of any one or more of embodiments 9-19, wherein the centrifuging step is performed without filtration.

Embodiment 21. The method of any one or more of embodiments 1-20, wherein step ii) comprises: seeding the mature adipocytes in a dedifferentiation container with a volume of growth media sufficient to completely fill the container; closing the dedifferentiation container to create a hypoxic environment; inverting the dedifferentiation container; incubating the inverted dedifferentiation container for a period of time sufficient for the mature adipocytes to adhere to the ceiling surface of the dedifferentiation container; and allowing the mature adipocytes to dedifferentiate into DFAT cells.

Embodiment 22. The method of embodiment 21, wherein the inverted dedifferentiation container is incubated for a period of about 10 days.

Embodiment 23. The method of any one or more of embodiments 1-22, wherein the one or more subculture populations of DFAT cells are cultured in step v) under conditions involving the manipulation of one or more parameters to preferentially encourage formation of the re-differentiated mature adipocytes, wherein the one or more parameters comprise: a coating to an interior surface of the container, one or more mechanical properties of a culture environment, growth media density, growth media osmolarity, growth media temperature, factors that encourage DFAT division rather than lipid production, and agents to control formation of non-adipocyte cells.

Embodiment 24. The method of any one or more of embodiments 1-23, wherein the first target biomass is reached upon achieving at least 70% confluence of DFAT cells.

Embodiment 25. The method of any one or more of embodiments 1-24, wherein the growth medium comprises only food-safe components.

Embodiment 26. The method of any one or more of embodiments 1-25, wherein the growth medium is serum-free.

Embodiment 27. The method of embodiment 26, wherein the growth medium does not comprise fetal bovine serum or fetal calf serum.

Embodiment 28. The method of any one or more of embodiments 1-27, wherein the culturing of step iii) and/or subculturing of step iv) comprises seeding growth medium with a concentration of at least 2000 cells/cm², and incubating until the cells reach at least 70% confluence.

Embodiment 29. The method of embodiment 28, wherein the subculturing of step iv) comprises seeding growth medium with a concentration of between about 5,000 cells/cm² to about 10,000 cells/cm², and incubating until the cells reach at least 75% confluence.

Embodiment 30. A fat-producing cell line created using the methods of any one or more of embodiments 1 to 29.

Embodiment 31. The fat-producing cell line of embodiment 30, wherein the fat-producing cell line comprises a total lipid accumulation of at least 80% of an intracellular volume when the cell line is at least 4 months old.

Embodiment 32. A cell line of re-differentiated fat-producing cells created using the methods of any one or more of embodiments 1 to 29, wherein the cell line is configured to survive in a suspension culture.

Embodiment 33. An imitation meat product for consumption comprising fat cells created using the methods of any one or more of embodiments 1 to 29, and an effective amount of an alternative meat source.

Embodiment 34. The imitation meat product of embodiment 33, wherein the fat cells represent up to about 5% of the imitation meat product.

Embodiment 35. The imitation meat product of embodiment 33 or 34, wherein the alternative meat source comprises cultured meat, a plant-based meat alternative, insect-derived proteins, fermentation-derived products, fungal proteins, or a combination thereof.

Embodiment 36. A method of creating an imitation meat product for consumption comprising: providing a plurality of mature adipocytes; incubating the plurality of mature adipocytes under conditions such that at least a portion of the plurality of mature adipocytes dedifferentiates, thereby generating a plurality of dedifferentiated fat cells (DFAT cells); culturing the plurality of DFAT cells in a growth medium until a first target biomass is obtained; subculturing the first target biomass in one or more subculture populations of DFAT cells; incubating the one or more subculture populations of DFAT cells under conditions such that at least a portion of the one or more subculture populations re-differentiates into one or more populations of re-differentiated mature adipocytes; and combining the one or more populations of re-differentiated mature adipocytes with an effective amount of an alternative meat source to create the imitation meat product.

Embodiment 37. The method of embodiment 36, wherein the alternative meat source comprises cultured meat, a plant-based meat alternative, insect-derived proteins, fermentation-derived products, fungal proteins, or a combination thereof.

Embodiment 38. A commercially scalable cell line for producing consumable animal fat, the cell line comprising re-differentiated fat-producing cells created by the methods of any one or more of embodiment 1-29, wherein the cell line is produced in a commercial-scale bioreactor.

Embodiment 39. A method of creating at least two distinct populations of dedifferentiated fat (DFAT) cells, the method comprising: obtaining adipose cells or a tissue explant that comprises adipose tissue; isolating a population of mature adipocytes from the adipose cells or tissue explant, wherein isolating the population of mature adipocytes comprises: adding the adipose cells or tissue explant to a digestion buffer in a container, wherein the digestion buffer comprises a collagenase; incubating the adipose cells or tissue explant in the digestion buffer until homogeneous digestion is achieved, optionally, at least about 30 minutes; centrifuging the adipose cells and digestion buffer to create a first floating adipocyte layer (FAL) above the digestion buffer and a pelleted tissue at the bottom of the container, wherein this step of centrifuging does not comprise filtration; removing the digestion buffer and pelleted tissue from the container; suspending the first FAL in growth media; centrifuging the first FAL and growth media to create a second FAL and a second pelleted tissue; removing the digestion buffer and the second pelleted tissue from the container; and suspending the second FAL in growth media, wherein the second FAL comprises the mature adipocytes; seeding the mature adipocytes in a dedifferentiation container with a volume of growth medium sufficient to completely fill the container; closing the dedifferentiation container to create a hypoxic environment; inverting the dedifferentiation container; incubating the inverted dedifferentiation container for a period of time sufficient for the mature adipocytes to adhere to a celling surface of the dedifferentiation container; allowing the mature adipocytes to dedifferentiate into DFAT cells; and establishing cell lines derived from two or more of the DFAT cells generated in step vii).

Embodiment 40. The method of embodiment 39, wherein the period of time for which the dedifferentiation container is incubated is about 10 hours.

Embodiment 41. The method of embodiment 39 or 40, wherein the collagenase is derived from microbial fermentation.

Embodiment 42. The method of any one or more of embodiments 39-41, wherein the collagenase comprises collagenase IV.

Embodiment 43. The method of any one or more of embodiments 39-42, wherein the digestion buffer comprises between 0.2% and 1% collagenase IV.

Embodiment 44. The method of embodiment 42 or 43, wherein the collagenase IV is from Clostridium histolyticum.

Embodiment 45. The method of any one or more of embodiments 39-44, wherein the tissue explant comprises subcutaneous fat extracted from a subject.

Embodiment 46. The method of embodiment 45, wherein the subject comprises a mammal, a bird, a fish, a reptile, a crustacean, or an amphibian.

Embodiment 47. The method of any one or more of embodiments 39-46, wherein the adipose cells do not comprise or were not derived from pluripotent stem cells, embryonic stem cells, or undifferentiated stem cells.

Embodiment 48. The method of any one or more of embodiments 39-47, wherein the adipose cells comprise primary mammalian cells, primary avian cells, primary fish cells, primary reptilian cells, primary crustacean cells, primary amphibian cells, or a combination thereof.

Embodiment 49. A method of obtaining avian dedifferentiated fat (DFAT) cells, the method comprising: i) providing adipose cells or a tissue explant comprising adipose tissue from an avian source; ii) introducing the adipose cells or tissue explant to a digestion buffer in a container, wherein the digestion buffer comprises collagenase IV; iii) incubating the adipose cells or tissue explant in the digestion buffer for at least about 30 minutes; iv) centrifuging the adipose cells and digestion buffer to create a first floating adipocyte layer (FAL) above the digestion buffer and a pelleted tissue at the bottom of the container; v) removing the digestion buffer and pelleted tissue from the container; vi) suspending the first FAL in growth media; vii) centrifuging the first FAL and growth media to create a second FAL and a second pelleted tissue; viii) removing the digestion buffer and the second pelleted tissue from the container; ix) suspending the second FAL in growth medium, wherein the second FAL comprises the mature adipocytes; x) seeding the mature adipocytes in a dedifferentiation container with a volume of growth medium sufficient to completely fill the container; xi) closing the dedifferentiation container to create a hypoxic environment; xii) inverting the dedifferentiation container; xiii) incubating the inverted dedifferentiation container for a period of time sufficient for the mature adipocytes to adhere to the ceiling surface of the dedifferentiation container; and xiv) allowing the mature adipocytes to dedifferentiate into DFAT cells.

Embodiment 50. The method of embodiment 49, wherein the adipose cells or tissue explant are added to the digestion buffer at a concentration of about 1 gram of adipose cells or tissue explant per 3 ml of digestion buffer.

Embodiment 51. The method of embodiment 49 or 50, wherein the collagenase IV is derived from microbial fermentation.

Embodiment 52. The method of any one or more of embodiments 49-51, wherein the digestion buffer comprises between about 0.2% and about 1% collagenase IV.

Embodiment 53. The method any one or more of embodiments 49-52, wherein the collagenase IV is from Clostridium histolyticum.

Embodiment 54. The method of any one or more of embodiments 49-53, wherein the avian source is a chicken, turkey, duck, pigeon, guinea fowl, or ostrich.

Embodiment 55. The method of any one or more of embodiments 49-54, wherein the adipose cells do not comprise or were not derived from pluripotent stem cells, embryonic stem cells, or undifferentiated stem cells.

Embodiment 56. The method of any one or more of embodiments 49-55, wherein the adipose cells comprise primary avian cells.

Embodiment 57. The method of any one or more of embodiments 49-56, wherein the centrifuging step is performed without filtration.

Embodiment 58. The method of any one or more of embodiments 49-57, wherein the period of time sufficient for the mature adipocytes to adhere to the ceiling surface of the dedifferentiation container is about 10 days.

Embodiment 59. The method of any one or more of embodiments 49-58, wherein the digestion buffer does not comprise collagenase I or collagenase II.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1. Isolation and Dedifferentiation of Mature Adipocytes into DFAT Cells—Exemplary Method

Adipose tissue is biopsied from an animal such as a mammalian or avian species, e.g., chicken. Adipocytes are isolated from the biopsied tissue, e.g., by mincing and enclosing in a tube containing a digestion buffer at a mass to volume ratio of, e.g., nearing or at 3 to 1. This ratio can be larger or smaller, potentially adjusting incubation time and temperature to achieve sufficient effect). During the adipocyte isolation process, an incubation period is typically included in which the tissue and digestion buffer are agitated at, e.g., a temperature of about 37° C. until the digestion is fully homogeneous (e.g., for approx. 30-45 mins). The temperature and duration of the incubation time may be varied relative to each other to shorten or extend the digestion period. Centrifugation at a low rotational speed, e.g., 1500-2000 RPM, is used to separate isolated adipocytes from the remaining stromal vascular fraction, and the buffer and the stromal vascular fraction (pelleted) are then removed from below the floating adipocyte layer (FAL) using, e.g., a hypodermic needle and syringe. The remaining, isolated adipocytes may be washed several times with cell culture media (e.g., resuspended in growth media and centrifuged).

The isolated mature adipocytes are transferred to a dedifferentiation container (e.g., a cell culture flask). Cell culture medium is added to the container, e.g., completely filling the container and occupying all of the remaining volume. The container can then be sealed to prevent gas exchange. The filled container is then incubated in a cell culture incubator at standard cell culture temperature (approx. 37° C.) and incubator conditions until a desired number of dedifferentiated fat (DFAT) cells have accumulated on the ceiling of the container, e.g., for 7 or more days.

At this point, the container may be inverted and the cells therein cultured conventionally (for example, to expand cell number). The DFAT cells can be removed from the culturing container and resuspended in culture medium. In some embodiments, the suspended cells are filtered through a strainer (e.g., pore size of approximately 50 micrometers to 1 millimeter) for the purpose of removing any remaining tissue particulates. Cells are propagated according to standard cell culture practices, e.g., involving two-dimensional adhesive culture, three-dimensional culture within a hydrogel, and/or various methods of floating culture. Cells are then passaged to a desired level of confluency. Culturing can include multiple passages of the DFAT cells to accumulate cell mass (e.g., at least 10, 15, 20, or 25 cell passages).

An exemplary cell culture medium for use in the steps of this example is 50% Ham's F12, 50% Dulbecco's Modified Eagle Medium, 10% Fetal Bovine Serum (FBS), and 1% PenStrep (penicillin-streptomycin). However, other basal culture media used for culturing of mammalian and avian cells can be used, including serum-free media. The digestion buffer can be 0.5% collagenase (5 mg/ml) in cell culture medium (though other concentrations may also be used, potentially adjusting incubation time and temperature to achieve sufficient effect). For mammalian adipose tissue, any of collagenase I, II, or IV can be used. For avian adipose tissue, collagenase IV or a collagenase having comparable enzymatic activity as described above in this disclosure should be used.

### Example 2. Culturing DFAT Cells to Produce Mature Adipocytes (Re-Differentiation) —Exemplary Method

DFAT cells (produced, for example, as described in the previous example) can be re-differentiated back to adipocyte cells. A differentiation medium of interest may then be used to induce changes to the morphology of the cells and generate a desired type of differentiated cells. For example, the introduction of a differentiation medium into the cellular environment can be used to induce a process of lipid production and accumulation within the cells, e.g., once they have been seeded at an appropriate density, and after exposure to the medium for a time period of, e.g., from 24 hours to 3 weeks. The accumulation media can contain the same reagents as the differentiation media or may contain additional agents as are known in the art for adipocyte differentiation (e.g., intralipid).

### Example 3. Cycling Dedifferentiated Fat Cells—Exemplary Method

Mature adipocytes generated, e.g., as described in Example 2, may be dedifferentiated, e.g., using the method described in Example 1, to regenerate dedifferentiated fat cells. The resulting dedifferentiated fat cells can then be redifferentiated, e.g., again using the method of Example 2. As such, the repeated rounds of alternating dedifferentiation and redifferentiation of the cells amounts to a method for cycling the cells, e.g., with respect to their differentiation state, morphology, and function. Such cycling can be performed for a substantial number of cycles, e.g., at least 1, 2, 3, 4, 5, 10, 15, 20, 25, 30, 35, 40, 45, or 50 cycles, or for an indefinite number of cycles.

### Example 4. Historical Methods for Isolating Mammalian Adipocytes are not Effective with Avian Species

Various published protocols for isolating mammalian adipocytes were attempted on fat tissue from avian species—specifically chicken fat tissue. Two previously published protocols on chicken fat tissue were also tested. The tested protocols included those described in Yagi 2004, Wei 2013, Watson 2014, Jumabay 2015, Shah 2016, Pu 2017, Lu 2018, Cui 2018, Zang 2020, Alexanderson 2020, Rogal 2020, Schopow 2020, Cho 2020, Panella 2021, and Hendaway 2021, the entire disclosures of each of which are herein incorporated by reference. Summaries of these protocols are provided below. The protocols were replicated with as much accuracy as possible, with minor variations in experimental execution (e.g., marginal variation in cell seeding density, reagent sources, and reagent concentration), with the exception of the protocols were performed using chicken fat tissue. Each protocol was tested multiple times.

Each of these protocols employs the use of a digestion buffer comprising collagenase I and/or II and employs filtration with centrifugation. Following digestion, ceiling culturing was performed. In no instances, were any viable adipocytes discovered on the ceiling of the cultures from these experiments. After digestion but prior to culturing, an aliquot of the digestion buffer/tissue mixture was observed by light microscopy. In each instance, digestion with collagenase I or II resulted in only lysed cells and, in some instances, triacylglycerol (TAG) aggregations (see representative micrograph images in FIGS. 3A-3B, respectively). To account for the possibility that our reagents were at fault, positive control experiments were performed using our reagents, including the collagenase I and II enzymes, on porcine tissue, which resulted in the expected successful isolation of viable cells.

After unsuccessfully replicating any of these published protocols with chicken fat tissue, we performed additional experiments aimed at creating a new protocol that would allow for the isolation of chicken derived DFAT cells. These experiments systematically investigated the concentrations of enzymes, digestion time of enzymes, potency of enzymes, and specific type of enzyme employed. Briefly, one series of experiments focused on the concentration gradients of Collagenase I and II. These working concentration gradients included many separate experiments such as gradients with 0.5%, 1.0%, 2%, and 4% enzyme, or gradients with 0.01%, 0.05%, 0.10%, 0.50%, and 1.00%. Similarly, experiments were performed assessing digestion time with observations and inoculations of ceiling flask with digest reacted for 20-100 minutes. These series of experiments resulted in either incomplete digestions at particularly low concentrations and/or reaction times, or cell lysis as was observed with published protocols that were tested.

It was hypothesized that collagenase I and II were digesting an off-target protein that induced chicken cell lysis. With this in mind, experiments were next performed with protease inhibitors of various concentrations added to the collagenase solutions to reduce hypothesized off-target effects of the collagenase I and II enzymes on avian cells. These experiments similarly failed to yield viable cells. Finally, experiments were performed with other enzymes, for example trypsin, in place of collagenase I or II. These modifications to existing protocols similarly failed to extract mature chicken adipocytes.

Thus, known dedifferentiation techniques, and obvious variations thereof, were not successful when employed on chicken fat tissue.

### 1. Yagi 2004

### Goal of Paper: Establishment of DFAT Line

Culture Protocol Excerpts/Summary: Cell culture. Isolation of primary mouse adipocytes was performed using a modified method as described by Sugihara et al. Ten-week-old male ddY mice were used to obtain inguinal fat pads. The fat tissues were minced and digested using 0.1% (w/v) collagenase at 37° C. for 1 hour. After filtration and centrifugation (135×g, for 3 min) of the digestion fluid, the floating primary adipocytes at the top layer and the SVF cells at the bottom of the tube were collected separately and washed. The adipose cells were then placed in a culture flask (SUMRON FLASK 50, Sumitomo Bakelite) filled with Dulbecco's modified Eagle's medium (DMEM) supplemented with 10% fetal bovine serum (FBS). The flask of the mature adipocytes was brimmed with the medium, providing the adipocytes with an air-free environment, and turned upside down, then incubated in a humidified 5% CO₂ atmosphere. The cells floated up in the medium and adhered to the top inner ceiling surface of the flask. When the cells became firmly attached and fibroblast-like shaped with no visible fat droplets after about a week, the flask was turned upside-down so that the cells were on the bottom. The medium was changed every 4 days until the cells were used for experiments. 3T3-L1 preadipocytes were cultured as previously described.

Both the preadipocytes derived from floating adipocytes and the 3T3-L1 cells were grown to semi-confluence for differentiations. Differentiation was then induced by changing the medium to DMEM supplemented with 10% FBS, 0.5 mM 3-isobutyl-1-methylxanthine (MIX), 0.25 μM dexamethasone (DEX), and insulin-transferrin-selenium-X supplement containing 51 g/ml insulin (Invitrogen). After 48 hours, the differentiation medium was replaced with DMEM supplemented with 10% FBS. The activity of glycerol-3-phosphate dehydrogenase (GPDH, EC1.1.1.8), a specific marker of terminal differentiation, was measured from the cell extracts as described previously. The amount of total protein of the cell extracts was also measured using the Lowry method.

Yagi 2004 Protocol details

DFAT Media Recipe: DMEM + 10% FBS
Collagenase Concentration: 0.10%
Filters used for Adipocytes: 100 μm
Centrifuge Step: 135 × g for 3 min
Ceiling Culture: Yes
Adipocyte Media Recipe: DMEM supplemented with 10% FBS, 0.5 mM 3-isobutyl-1-
methylxanthine (MIX), 0.25 μM dexamethasone (DEX), and insulin-transferrin-selenium-X
supplement containing 5 μg/ml insulin (Invitrogen).
Insulin Concentration: 5 μg/ml

### 2. Wei 2013

### Goal of Paper: Bovine DFAT Isolation

Culture Protocol Excerpts/Summary: Subcutaneous fat samples from an Angus steer were washed with PBS several times before being placed into an appropriate culture hood. About 15 grams fat tissue was collected from trimmed samples into a 100 mm dish. Five grams of fine cut fat (about 3 mm) fragments were transferred into each fresh sterile 50 ml centrifuge tube (n=3). To this, pre-warmed collagenase type I (Gibco) was added. The tissue-collagenase mixture was incubated in a continuously shaking 37° C. water bath for 1 hour. Following collagenase digestion, contents of the tubes were filtered through a 1000 μm sterile plastic mesh into fresh sterile 50 ml centrifuge tubes. Centrifugation at 186×g for 10 min was performed to separate the collagenase digested tissue into three layers; the supernatant (top layer) containing adipocytes; the infranatant (middle layer) containing the collagenase; and the pellet at the bottom formed from the stromal vascular fraction. The top layers were subsequently transferred into 3 fresh sterile 50 ml centrifuge tubes each containing 20 ml DMEM/F12+10% HS. Centrifugation at 186×g for 10 min was performed, followed by two repeated washing steps. After filtration, centrifugation and washing, the floating top layers containing adipocytes were collected and used in the first trial plate ceiling culture (FIG. 6). Two combinations (three 35 mm×10 mm dishes [ceiling; Falcon]+one 100 mm×20 mm dish [container; Nunc] and one 100 mm×10 mm lid [ceiling; Nunc]+one 150 mm×20 mm dish [container; Nunc]) were chosen to initiate plate ceiling culture; one T-12.5 cm² flask (Falcon) ceiling culture served as control (FIG. 6). An inverted phase contrast microscope with 4× and 10× objectives was available in the hood to mark lipid containing cells/remove contaminating cells. Adipocytes were transferred into the previously mentioned cell culture plates and flask. Culture medium was then carefully added. The inverted small dishes and the lid were placed into the bigger dishes, while the medium-filled flask was tightly closed and inverted. On days 3, 4, and 5 of cell isolation the three small dishes were re-inverted and small amount of basal media (DMEM/F12+10% FBS) was added to check the mature adipocyte attaching efficiency. The big lid and the flask were re-inverted on day 5. Mature adipocyte attaching efficiency was measured in three methods (dish ceiling culture, lid ceiling culture and flask ceiling culture).

Wei 2013 Protocol details

DFAT Media Recipe: DMEM/F12 + 10% FBS
Fat Starter: 5 grams
Collagenase Type: I (Gibco)
Filters used for Adipocyte: 1000 μm
Centrifuge Step: 186 × g for 10 min in 37° C. collagenase solution followed by aspiration of top
layer of supernatant and then 186 × g for 10 min in 37° C. in DMEM
Ceiling Culture: Yes
Adipocyte Media Recipe: Adipocyte DM-2 contained DMEM/Ham's F-12 (1:1, v/v), HEPES
pH 7.4, FBS, biotin, pantothenate, human insulin, dexamethasone, isbutylmethylxanthine,
PPARg agonist, penicillin, streptomycin, and amphotericin B
Days required for attachment: 1-7 days

### 3. Watson 2014

### Goal of Paper: ADSC and DFAT Comparison

Culture Protocol Excerpts/Summary: Adipocytes, obtained by the top layer of cells following centrifugation of the digested fat tissue, were placed in 75-mm culture flasks filled with the a-MEM with 10% FBS and a modified ceiling culture method was used to isolate adhered adipocytes at the end of 2 weeks followed by another 2-3 weeks in bottom culture (or longer) in hypoxia to improve the dedifferentiation process

Watson 2014 Protocol details

DFAT Media Recipe: alpha-MEM with 10% FBS
Collagenase Type: I (Worthington)
Collagenase Concentration: 0.075% in modified PBS
Filters used for Adipocyte: 100-um cell strainer (BD Falcon), second round: 70 um cell strainer
(BD Falcon)
Centrifuge Step: Mature adipocytes were collected by centrifugation in a 50-mL conical tube
(400 × g, ambient temperature, 5 min) and removed as the supernatant, second round: 300-500 ×
g, 5 minutes
Ceiling Culture: Yes
Adipocyte Media Recipe: Adipocyte DM-2 contained DMEM/Ham's F-12 (1:1, v/v), HEPES
pH7.4, FBS, biotin, pantothenate, human insulin, dexamethasone, isbutylmethylxanthine,
PPARg agonist, penicillin, streptomycin, and amphotericin B.
Days required for attachment: a modified ceiling culture method was used to isolate adhered
adipocytes at the end of 2 weeks followed by another 2-3 weeks in bottom culture

### 4. Jumabay 2015

### Goal of Paper: DFAT Review

Culture Protocol Excerpts/Summary: Adipocytes intended for DFAT cell generation are floated on top of medium in culture dishes or plastic tubes to let remaining non-adipocytes detach and sink to the bottom and be discarded after centrifugation. Adipocytes from the top creamy layer (30-50 L) are subsequently transferred to 6-well plates fitted with 70 μm-filters and incubated for 5 days in culture medium. DFAT cells derived from the adipocytes sink through the filters and attach to the bottom of the dishes. The filters with remains of the adipocytes are removed after 5 days.

Jumabay 2015 Protocol details

DFAT Media Recipe: DMEM supplemented with 20% fetal bovine serum
Fat Starter: 1 gram
Collagenase Type: Type I
Filters used for Adipocytes: 70 μm
Centrifuge Step: 135 × g for 3 min
Ceiling Culture: No

### 5. Shah 2016

### Goal of Paper: DFAT Review

Culture Protocol Excerpts/Summary: Adipose tissues were minced and then underwent enzymatic digestion with collagenase type II. After centrifugation, the resulting pellet containing the SVF was cultured as ASCs and the floating adipocytes were collected, washed, and transferred to an inverted cell culture flask filled with cell culture medium. The cells floated in the media and adhered to the ceiling of the flask. After the cells were firmly attached and fibroblast-like cells were observed, the flask was re-inverted to culture the attached cells as DFAT cells.

Shah 2016 Protocol details

DFAT Media Recipe: high-glucose DMEM, 2 mM L-glutamine, 50 mg/ml gentamycin, and
10% FBS
Collagenase Type: II
Ceiling Culture: Multiple methods stated
Days required for attachment: 7

### 6. Pu 2017

### Goal of Paper: PPARy Agonist for DFAT

Culture Protocol Excerpts/Summary: Isolated by rinsing a 1-2 gram of adipose tissue with pre-warmed Dulbecco's phosphate-buffered saline (DPBS) with antibiotic-antimycotic (Invitrogen), removing any visible blood clots and connective tissue using sterile forceps and then minced into small pieces. Collagenase-I (C0130, Sigma, 1 mg/ml) was used to digest tissue for 40 min in a 37° C. water bath. The mixture was filtered through a mesh filter (250 μm, U-CMYBK-250, Component Supply Company, Fort Meade, USA) and centrifuged at 1,200 RPM for 5 min. The cell pellet was washed with fresh omental preadipocyte medium and seeded into 6-well plates. After six days of culture, cells were frozen (Preadipocyte Cryopreservation Medium, FM-1-100, Zen-Bio) and stored in liquid nitrogen until further use.

Pu 2017 Protocol details

Fat Starter: Sheep, 1-2 grams
Collagenase Type: I (C0130, Sigma, 1 mg/ml)
Filters used for Adipocyte: 250 μm, U-CMYBK-250, Component Supply Company
Centrifuge Step: 1,200 RPM for 5 min.
Ceiling Culture: Yes
Adipocyte Media Recipe: basal medium supplemented with biotin (33 μM), pantothenate, (17
mM), insulin (1 μg/ml), dexamethasone (1 μM) and 3-isobutyl-1-methylxanthine, (IBMX, 0.5
mM)
Insulin Concentration: insulin (1 μg/ml), and increased to 10 μg/ml

### 7. Lu 2018

Goal of Paper: ADSC Extraction and Differentiation from Chicken

Culture Protocol Excerpts/Summary: Isolation and culture of ADSCs. Adipose tissue samples were collected from 20-day-old broiler embryos under aseptic conditions. The adipose pads were washed with PBS supplemented with 100 IU/ml penicillin and 100 μg/ml streptomycin to remove any other cells, including hemocytes and endothelial cells. The adipose pats were fully chopped into small pieces and subsequently incubated with 0.2% (m/v) collagenase I in PBS at 37° C. for 40 min. The enzymatic activity was neutralized by adding an equal volume of DMEM/F12 containing 5% (v/v) FBS. The cell suspension was filtered through a 74-mm-mesh sieve and then centrifuged at 200×g for 8 min at room temperature. The precipitate was resuspended in complete growth medium composed of DMEM/F12, 10% (v/v) FBS, 2 mM L-glutamine, 100 IU/ml penicillin, 100 g/ml streptomycin and 10 ng/ml bFGF. Cells were seeded into petri dishes at a density of 1.0×10⁵/ml, and cultured at 37° C. with 5% CO₂. After 24 hours, the dishes were washed with PBS to clean out any non-adherent cells, including pericytes, blood cells, endothelial cells and preadipocytes. The cells were referred to as ‘passage 0’ (P0) when their confluence reached 80%. Subsequently, the cells were sub-cultured at the ratio of 1:2 after standard trypsin digestion. This generation was referred to as P1. The cells became purified with increasing passages, and were then harvested for other relevant trials.

Adipogenic differentiation of ADSCs. ADSCs were divided into a control group and an induction group. When the confluence of cells reached 70-80%, the experimental group was treated with adipogenic medium consisting of DMEM/F12 containing 10% FBS, 200 μM indomethacin, 1 mM dexamethasone, 10 M insulin transferrin and 0.5 mM IBMX. The control group was cultured in complete growth medium without the factors mentioned above. The medium was replaced every 2 days. After 1 week, the two groups were evaluated for accumulation of intracellular lipid using oil red O staining for 40 min at room temperature and expression of adipogenic cell-specific genes by RT-PCR.

Lu 2018 Protocol details

Collagenase Type: Type I
Collagenase Concentration: 0.20%
Adipocyte Media Recipe: DMEM/F12 containing 10% FBS, 200 μM indomethacin, 1 mM
dexamethasone, 10 μM insulin transferrin and 0.5 mM IBMX.
Insulin Concentration: 10 μM

### 8. Cui 2018

Goal of Paper: Co-Culture System with Myocytes and Adipocytes

Culture Protocol Excerpts/Summary: The main protocol was as follows: The pectoralis major was washed 2 times with phosphate-buffered saline (PBS) containing 1% penicillin-streptomycin. After the blood vessels and connective tissue were removed, the sample was finely minced to 1 mm³ with the use of a pair of scissors. The samples were then digested in Dulbecco's Modified Eagle's Medium (DMEM)/F12(1:1) containing 0.1% Type I collagenase in a water bath, with continuous shaking at 37° C. for 60 min. After digestion was terminated, the cell suspension was filtered and centrifuged with 600×g for 15 min. The rock-bottom cell precipitation was re-suspended with DMEM/F12(1:1) medium containing 10% FBS and then seeded in a 100-mm dish. After cells were incubated with a 5% C02 at 37° C. for 2 hours, the medium containing unattached cells was transferred to a new dish, and purified muscle satellite cells (MSC) were obtained. The top mature adipocytes were collected and placed into a 25 cm² cell culture flask, and the flask was completely filled with DMEM/F12(1:1) medium containing 10% FBS and inverted to keep the floating mature adipocytes adhering the bottom of the flask in a 37° C. incubator with 5% CO₂. After 3 to 5 days, the mature intramuscular adipocytes (MIA) gradually released lipid by exocytosis, and then the medium was replaced and the flask was re-inverted to allow the proliferation of preadipocytes.

Cui 2018 Protocol details

Fat Starter: Chicken
Collagenase Type: Type I
Collagenase Concentration: 0.1%
Centrifuge Step: 600 × g for 15 min
Adipocyte Media Recipe: DMEM/F12(1:1) medium containing 10% FBS

### 9. Zang 2020

### Goal of Paper: Insulin Regulation

Culture Protocol Excerpts/Summary: Mice epididymal white adipose tissues were removed and digested in 0.1% (v/v) type I collagenase solution (containing 0.4% BSA, v/v) in a 37° C. water bath with shanking at 100 RPM for 35 min. After adding cell growth medium (DMEM/F12 containing 10% FBS (v/v), 100 U/ml penicillin and 100 μg/ml streptomycin) was used to stop the digestion. The suspension was centrifuged at 200×g for 10 min, and then the cell pellet was resuspended in a cell growth medium, was filtered through a 100-μm strainer, and was seeded in 25-cm² flasks. Mesenchymal stromal cells do not easily to attach to the bottom of a petri dish after seeding when compared to fibroblasts, but they are more sensitive to trypsin when passage digestion takes place, and they are easy to de-attach from dish. Therefore, we use differential adherence and incomplete digestion methods to improve the purity of mesenchymal stromal cells until they were at passage 4 (P4) to be sub-cultured into 12-well plates. These preadipocytes were then used for further experiments.

Zang 2020 Protocol details

DFAT Media Recipe: DMEM/f12 containing 10% FBS, 100 U/ml penicillin and 100 μg/ml
streptomycin
Collagenase Type: Type I (containing .4% BSA)
Filters used for Adipocytes: 100 μm
Centrifuge Step: 200 × g for 10 min
Insulin Concentration: 17 nM

### 10. Alexanderson 2020

Goal of Paper: Mature Adipocyte Isolation from Tissue

Culture Protocol Excerpts/Summary: Place the human adipose tissue in a 15 cm diameter petri dish and add a small volume of Medium 199 to keep it moisturized during the dissection. Working with pieces of adipose approximately the size of a golf ball, grasp large fibrotic vessels with tweezers and gently release the adipocytes by scraping the adipose with the back of closed scissors. Discard the large pieces of fibrotic tissue. Weigh the trimmed fat.

Collagenase Treatment: Add collagenase to the digestion buffer at a concentration of 1 mg/mL. Sterile filter the solution using a 0.22 μm sterile filter. Three mL of digestion buffer per gram of fat is recommended). Move approximately 10 grams of adipose tissue to a 15 cm diameter petri dish and mince the fat carefully using a pair of curved scissors until it becomes a smooth homogenous mixture and there are no large pieces of adipose left. Repeat the process until all of the fat has been processed. The adipose pieces should be small enough so that they can be pipetted using a wide-bore pipette tip. If the pieces are too large, digestion times can be extended, which can compromise cell viability. Digest the tissue at 37° C. in a shaking incubator with constant agitation at 150 RPM for 30-45 min. After 30 min, check on the process every 5 min to avoid over-digestion. The digestion is complete when the adipose solution is homogenous without any large pieces and has an apricot color.

Alexanderson 2020 Protocol details

Fat Starter: 100 grams
Collagenase Type: Type II
Collagenase Concentration: concentration of 1 mg/ml and 3 to 1 ml of digestion solution to
grams of fat + 500 mL of collagenase buffer containing 1 × HBSS + CaCl₂ + MgCl₂, 2% BSA,
and 450 mL of water
Filters used for Adipocytes: 250 μm mesh filter inside the funnel
Ceiling Culture: Membrane seeded inverted culture using well plate
Adipocyte Media Recipe: Dulbecco's modified Eagle's medium/Ham's nutrient mixture F12
(DMEM/F12), 10% fetal bovine serum (FBS), 1% penicillin-streptomycin (penn/strep) and 20
nM insulin
Insulin Concentration: 20 nm

### 11. Rogal 2020

### Goal of Paper: WAT-On-a-Chip

Culture Protocol Excerpts/Summary: Adipose tissue was rinsed twice with Dulbecco's phosphate buffered saline with MgCl₂ and CaCl₂ (PBS+; Sigma-Aldrich Chemie GmbH), and visible blood vessels, as well as connective-tissue structures, were thoroughly removed. The remaining adipose tissue was cut into fine pieces of approximately 1 cm³, and digested with a collagenase solution [(0.13 U/ml collagenase type NB4 (Serva Electrophoresis GmbH, Heidelberg, Germany) in Dulbecco's Modified Eagle Medium (DMEM/Ham's-F12; Thermo Fisher Scientific, Waltham, USA), with 1% bovine serum albumin (BSA; Sigma-Aldrich Chemie GmbH)] for 60 min at 37° C. on a rocking shaker (50 cycles/minute; Polymax 1040, Heidolph Instruments GmbH & CO. KG, Schwabach, Germany). Next, the digested tissue was passed through cell strainers (mesh size: 500 μm), and subsequently washed three times with DMEM/Ham's-F12. For each washing step, adipocytes and medium were mixed, and left to rest for 10 min to allow separation of the buoyant adipocytes and the medium; this was followed by aspiration of the liquid media from underneath the packed layer of adipocytes.

Rogal 2020 Protocol details

Fat Starter: Human
Collagenase Type: collagenase type NB4 (Serva Electrophoresis GmbH, (contains I and II)
Collagenase Concentration: 0.13 U/ml
Filters used for Adipocyte: 500 μm
Adipocyte Media Recipe: subcutaneous Adipocyte Maintenance Medium (AM-1; BioCat
GmbH)

### 12. Schopow 2020

### Goal of Paper: Tissue Slice Culture

Culture Protocol Excerpts/Summary: Adipose tissue slices were transferred onto cell culture inserts with a pore size of 0.4 μm (Millipore, Merck, Darmstadt, Germany) placed in six-well plates (Corning, New York, USA), and cultivated on a liquid-air-interface in a humidified incubator at 35° C. and 5% CO₂ (FIG. 1). Each well contained 1 ml culture medium under the membrane inserts supplying the tissue via diffusion.

Schopow 2020 Protocol details

Fat Starter: 5 × 5 × 10 mm pieces, 350, 500, and 750 μm thick slices
Adipocyte Media Recipe: DMEM, insulin-transferrin-selenium mixture (1:100, ITS, Sigma
Aldrich, Saint Louis, USA), and Penicillin/Streptomycin (1:100, PenStrep, Gibco). Fetal bovine
serum (1:10FBS, Gibco) or human tumor necrosis factor alpha (50 ng/ml, TNFα, Pepro Tech)
Insulin Concentration: 1 to 100

### 13. Cho 2020

### Goal of Paper: Fat Disassociation

Culture Protocol Excerpts/Summary: Incubate the epididymal fat pad in five milliliters of HBSS supplemented with 3% BSA in a 50 milliliter conical tube for 15 minutes. After the incubation, centrifuge the tube at 150×g for seven minutes at four degrees Celsius. Remove the floating epididymal fat pad from the conical tube and finely mince it with clean scissors. Add 200 milliliters of 2% collagenase solution to 3.8 milliliters of HBSS in a 13 milliliter culture tube. Then add the minced tissue and incubate it in a rotating incubator at five RPM for one hour at 37 degrees Celsius. Transfer the entire contents into a 50 milliliter conical tube and add 10 milliliters of neutralization medium. Mix gently by inverting the tube two to three times. Prepare another 50 milliliter conical tube fitted with a 70 micrometer cell strainer and filtered the digested tissue into the new tube. Transfer the flow through to a 15 milliliter tube and centrifuges at 350×g per 10 minutes at four degrees Celsius. Carefully remove the supernatant and resuspend the cell pellet with five milliliters of DPBS. Then centrifuge the tube for another 10 minutes. Carefully remove the supernatant.

Cho 2020 Protocol details

Fat Starter: Mouse, unknown quantity
Collagenase Type: Unknown
Collagenase Concentration: 200 mL of 2% collagenase solution to 3.8 milliliters of HBSS
Filters Used for Adipocyte: 70 micrometer cell strainer
Centrifuge Step: 350 × g per 10 minutes at four degrees Celsius

### 14. Panella 2021

### Goal of Paper: Serum Free Cell Culture

Culture Protocol Excerpts/Summary: Adipose tissue was washed with DPBS with Ca²⁻ and Mg²⁺ (Bioconcept, Allschwil, Switzerland) in a Gosselin sterile container (Corning, New York, USA), cut into smaller pieces, and homogenized with an immersion blender. The fat was dissociated by digestion with collagenase type B, animal origin free (Worthington, Biochemical Corp., Lakewood, NJ), at a final concentration of 0.28 Wunsch U/mL for 45 min at 37° C. under constant, but gentle agitation. The dissociated cells were subsequently filtered through a 100 μm and then through a 40 μm sieve cell strainer (Becton Dickinson, Franklin Lakes, NJ). All centrifugation steps were completed at 600×g for 10 min at room temperature. At the end of the extraction procedure, the cellular pellet was resuspended in PBS without Ca²⁺ and Mg²⁺ (Biowest, Nuaille, Frace) with 1% injectable human albumin (CLS Behring, Bern, Switzerland) or in the cell culture medium. Cells were seeded at a density of approximately 30,000 ASCs/cm² in Fibronectin coated vessels (Corning, New York, USA) and kept in a humidified incubator at 37° C. and 5% CO₂. This initial passage of the primary cell culture was referred to as passage 0 (P0). Details can be found in the Supplementary Information (S2: Characterization of the cells of the SVF from adipose tissue, S3: Initial plating of cells of the SVF for expansion, and Figure S4). Cells were maintained in culture until they achieved 75-90% confluence. They were then either collected and cryopreserved or replated in new Fibronectin coated tissue culture vessels at a density of 5000-10,000 ASCs/cm². The medium was changed every 2-3 days, always keeping 20-50% of the conditioned medium, until the cells reached confluency between 75 and 90%. Cells were detached with TrypLEe Select (Life Technologies, Thermo Fisher Scientific, Waltham, USA) for 2 min at 37° C., then washed with PBS supplemented with 1% injectable human albumin and pelleted by centrifugation at 400×g for 5 min

Panella 2021 Protocol details

Fat Starter: Human
Collagenase Type: collagenase type B, animal origin free (Worthington)
Collagenase Concentration: 0.28 Wünsch U/mL for 45 min at 37° C. under constant, but gentle
agitation
Filters used for Adipocyte: 100 μm and then through a 40 μm sieve cell strainer (Becton
Dickinson
Centrifuge Step: 600 c g for 10 minutes at room temperature
Adipocyte Media Recipe: proprietary defined serum- and xeno-free cell culture medium,
“UrSuppe”. The medium contains only defined molecules, some recombinant human growth
factors, and injectable human albumin in the μg range

### 15. Hendaway 2021

Goal of Paper: Effect of anatomical site

Culture Protocol Excerpts/Summary: Minced adipose tissue was placed in a shaking bath for 1 hour at 37° C. in Hank's balanced salt solution (HBSS) containing 0.1% (w/v) collagenase type 1 (1 mg/mL; Gibco by Life Technologies, Waltham, MA, USA). Cold HBSS was added to neutralize the collagenase enzyme activity. Following centrifugation (800×g, 10 min), we obtained a high-density cell pellet. Solid aggregates were removed via filtration through a 100 μm filter (BD Falcon, Bedford, MA, USA). A 1 mL lysis buffer (Promega, Mannheim, Germany) was used to resuspend the pellet and lyse RBCs. Finally, the cell pellet was resuspended in Dulbecco's Modified Eagle Medium (DMEM), containing 4500 mg/L D-glucose, 110 mg/L Sodium Pyruvate, 4 mM L-Glutamine, 10% Fetal Bovine Serum (FBS), 1% non-essential amino acids, and 1% Penicillin-Streptomycin (P/S), as a basal culture medium. Unless otherwise specified, we purchased all chemicals from Sigma-Aldrich.

Hendaway 2021 Protocol details

Culture Protocol Excerpts/Summary:
Fat Starter: Rat
Collagenase Type: Type I (Gibco)
Collagenase Concentration: 1 mg/mL
Filters used for Adipocytes: 100 μm
Centrifuge Step: 200 × g for 10 min
Adipocyte Media Recipe: DMEM + 10% FBS

### Example 5. Isolation and Dedifferentiation of Avian and Mammalian Adipose Tissue-Derived Stem Cells to Produce DFAT Cells

In view of the results obtained in the prior example, we developed a methodology for the isolation and dedifferentiation of adipose tissue-derived stem cells to DFAT cells from livestock sources that could be used in particular for avian sources. The method is described in detail for chicken adipose but was also tested across multiple avian sources as well as a mammalian source, as described below.

To begin this process, we isolated subcutaneous adipose tissue, adhering to stringent sterile protocols and utilizing the appropriate personal protective equipment. We excised adipose tissue from recently sacrificed livestock, ensuring the tissue was as soon as possible post-sacrifice. With precision, a gram of this adipose tissue was weighed and minced. Minced adipose tissue (1 gram) was placed into a Falcon™ tube containing 3 ml of the digestion buffer. The digestion buffer contained 5 mg/ml collagenase type IV in a basal culture medium. The basal culture medium used was 50% Ham's F12, 50% Dulbecco's Modified Eagle Medium, 10% Fetal Bovine Serum (FBS), and 1-5% antibiotics. The Falcon™ tubes were placed in an incubator set at 37° C., gently rotating on a tube rack for a duration of from 30 to 45 minutes.

Post-incubation, the Falcon™ tubes were centrifuged (5 min, 300×g) to separate the isolated floating adipocyte layer (FAL) from the digestion buffer and the pelleted stromal vascular fraction (SVF). In a biosafety cabinet, the digestion buffer and SVF were carefully removed from beneath the FAL using a hypodermic needle and syringe. The FAL was then resuspended in several milliliters of basal media. The tubes were once again subjected to centrifugation at the same rotational speed, facilitating the extraction of the basal media used for resuspension. This washing procedure was repeated to ensure removal of less buoyant contaminant cells (e.g., adipose derived stem cells (ADSCs)).

To dedifferentiate the cells, the remaining FAL was transferred to T25 flasks. The flasks were completely filled with basal media. We determined that it was important to keep the cells in hypoxic conditions, thus the flasks were sealed with filterless caps. The filled flasks were then consigned to a 37° C. incubator, and left undisturbed for a period ranging from 5 to 14 days until the desired fibroblastic morphology was observed on cells adherent to the ceiling.

Upon conclusion of the incubation period, the flasks were carefully opened, and the media gently drained. The flasks were inverted, and the surface that had initially served as the ceiling was thoroughly washed with phosphate-buffered saline (PBS). Subsequently, the surface was exposed to trypsin and subjected to pipetting, effectively removing any dedifferentiated fat cells that had adhered to the flask ceiling during the incubation period. In some instances, the protocol could be modified to use a cell scraper or other well-known means to de-adhere the cells.

The trypsin was inactivated by mixing a ratio of 5 ml of FBS containing media per 2 ml of trypsin-cell solution. This solution was next filtered through a 70-micron pore cell strainer. Following centrifugation (300×gravity, 5 min), the dedifferentiated fat (DFAT) cells were delicately resuspended in basal media and transferred to a designated cell culture vessel for further analysis and experimentation. In some instances, the protocol could be modified to skip this inactivation step and the cells could be pelleted directly and then resuspended in media.

### Example 6. Confirmation of Lipid Accumulation Identity

Lipid accumulation is a marker of DFAT cells as compared to mature adipocytes. Lipid accumulations are most conveniently and reliably determined by light microscopic assessment by one skilled in the art. Thus, to confirm that the subcellular objects observed by light microscopy in the DFAT cells obtained from the previous example corresponded to lipid accumulation, Nile Red and Oil Red-O staining were performed (as described by the manufacturer and in published protocols). Oil Red-O staining was performed on fixed cells both on slides and in extracts measured on an optical Tecan plate reader. Nile Red staining was performed in conjunction with the nuclear stain Hoescht 3342 on live cells, with both dyes applied to cells following the manufacturer's instructions after basal media was washed and replaced with PBS in the 6-well plate well in which cells were actively growing. Images provided were taken with a Nikon epifluorescent microscope at room temperature.

Avian adipocytes and DFAT cells obtained using these methods are shown in the pictorial micrographs of FIGS. 4A-4F with relevant molecular dyes to validate what brightfield visible structures are. Briefly, FIG. 4A shows healthy, dissociated adipocytes viewed in floating suspension under brightfield illumination. FIG. 4B shows DFAT cells derived from the mature adipocytes of FIG. 4A stained with the lipid dye Nile Red. FIG. 4C Shows an enlarged view of these same Nile Red stained DFAT cell from FIG. 4B wherein Nile Red staining highlights fibroblastic pseudopodia and ‘loculus-dividing’ lipid droplets and dividing nucleus. FIG. 4D Shows the brightfield illuminated view of the DFAT cells derived from the mature adipocytes in FIG. 4A, at the same imaging region as FIG. 4B. FIG. 4E shows derived DFAT cells from the mature adipocytes in FIG. 4A additionally stained with the nucleic acid dye Hoechst 33432. FIG. 4F Shows an enlarged view of a single DFAT cell undergoing mitosis, wherein Hoechst 33432 nuclear staining highlighting mitosis as nuclear material shuttling into splitting nuclei, indicating this cell is in anaphase or telophase of mitosis.

Thus, unlike all other previously disclosed methodologies, which work only on mammalian species, the above method can be used to dedifferentiate DFAT cells from chicken adipose cells.

### Example 7. Isolation and Dedifferentiation of DFAT Cells from Diverse Species

To test if the above protocol is species agnostic, butchered livestock samples from chicken, turkey, duck, pigeon, and rabbit were subjected to the protocol described in Examples 1 and 5 without modification and found to be effective in all tested species (see FIG. 8). The cladogram computed by sequence alignment of 18S rRNA establishes the specific divergence from a last common ancestor for each of these species in units of millions of years. These species represent a large cross section of evolutionary space with examples of all common avian livestock groups and a representative mammal. Thus, in contrast to current methodologies, this method is widely applicable to both mammalian and avian cells.

### Example 8. Differentiation of the DFAT Cells

To perform cycling, DFAT cells at a passage level ranging from 1 to 7 were cultivated until they reached a confluence level of 70-90% as assessed by light microscope. These cells were then carefully disadhered from the culture vessel using trypsin, washed, and subsequently resuspended in Adipocyte Differentiation Media (a basal culture medium containing high glucose, dexamethasone, 3-isobutyl-1-methylxanthine, rosiglitazone, and insulin).

The cell suspension was then introduced into a culture vessel containing the Adipocyte Differentiation Media, with cells being seeded at a density of approximately 7,000 to 15,000 cells per square centimeter (the cell density could also be lower or higher). Throughout the experimental procedure, the Differentiation Media was refreshed every 48 hours (though media changes could be made more frequently or, in some instance, less frequently).

FIGS. 5A-5C provide a sequential series of pictorial micrographs showing a timelapse of DFAT re-differentiation in these cells using the disclosed method for re-differentiating DFAT cells into adipocytes by means of a differentiation media. FIG. 5A shows DFAT cells following two days of exposure to differentiation media. FIG. 5B shows a population of DFAT cells after five days of exposure to differentiation media. A notable increase in accumulation of lipid droplets was observed, and the cells were developing adipocyte morphology. Finally, FIG. 5C is a pictorial micrograph taken at two weeks after the introduction of differentiation media. As can be seen, at this two-week time point, the cells contained large lipid droplets that are similar in size to those of mature adipocytes, and the cells assumed a more rounded morphology, which is characteristic of mature adipocytes. In some cases, the lipid droplet size in certain cells at two weeks following introduction of differentiation media exceeded that traditionally observed in mature adipocytes.

### Example 9. Cycling

Once the detection of a sufficiently substantial lipid accumulation, typically reaching around 20 microns in size and occurring over a period of approximately 5 to 25 days, the cells were considered to have differentiated into adipocytes. Subsequently, these differentiated cells were passaged and re-seeded in basal media. This process induced a reduction in the size and number of lipid droplets, ultimately leading to the cells reverting to a dedifferentiated morphology. In this manner, the cells were subjected to a cyclical process, enabling the systematic examination of their differentiation and dedifferentiation.

To demonstrate that cells are able to repeatedly transition from a proliferative, fibroblastic, DFAT state to the lipid accumulating adipocyte state this cyclical culturing of cells in either basal or differentiation media, as here described, was conducted. FIGS. 6A-6D show pictorial micrographs that demonstrate a timelapse of the DFAT cycling process. FIG. 6A provides micrographs at two days following the introduction of differentiation media to the cellular environment. The gray arrow indicates a large lipid droplet that has formed within one of the cells. After obtaining the micrograph of FIG. 6A, the differentiation medium was removed, and FIG. 6B shows a pictorial micrograph of cells two days later. As shown, after removal of the differentiation medium, the cells in FIG. 6B exhibit less lipid accumulation and assume a more elongated morphology that is characteristic of pluripotent cells. The cells were then passaged and differentiation media reintroduced. FIG. 6C shows a pictorial micrograph of the cells one day after passage and the re-introduction of differentiation media. As seen in FIG. 6D, lipid accumulation was observed (as indicated by the gray arrow) after only two days following the re-introduction of the differentiation medium.

To demonstrate cycled DFAT cells were not only capable of re-acquiring the lipid accumulating state, but could also still become proliferative, cells were repeatedly grown to confluence in basal media and passaged as is typical for cell expansion. To demonstrate these cycled DFAT cells could reliably be re-differentiated into mature adipocytes, the above protocol involving seeding 7,000 to 15,000 cells per square centimeter in continually refreshed differentiation media was performed.

FIGS. 7A-7D provide sequential pictorial micrographs to demonstrate the cycling of re-differentiated DFAT cells into adipocytes, followed by de-differentiation again to a the multi-potent morphology under one embodiment of the present disclosure. FIG. 7A provides a photograph of the DFAT cells one day after seeding. Differentiation media was then introduced, and, as seen in FIG. 7B, lipid droplets (as indicated by the gray arrow) accumulated within the cells and the cells neared adiopogeneic maturity after only five days following the introduction of differentiation media. Cells were then passaged. As shown in FIG. 7C, nearly all of the lipid accumulation was lost after one day of the passage phase. Cells then remained in growth media, and ten days after passage (FIG. 7D), the cells assumed an elongated, fibroblastic morphology.

References identified in this disclosure, each of which is incorporated by reference in its entirety herein, include:
Alexandersson, Ida, Matthew J. Harms, and Jeremie Boucher. “Isolation and Culture of Human Mature Adipocytes Using Membrane Mature Adipocyte Aggregate Cultures (MAAC).” Journal of Visualized Experiments, no. 156 (Feb. 13, 2020): 60485. doi.org/10.3791/60485.
Asgar, M. A., A. Fazilah, Nurul Huda, Rajeev Bhat, and A. A. Karim. “Nonmeat Protein Alternatives as Meat Extenders and Meat Analogs.” Comprehensive Reviews in Food Science and Food Safety 9, no. 5 (September 2010): 513-29. doi.org/10.1111/j.1541-4337.2010.00124.x.
Bhat, Zuhaib F., James D. Morton, Susan L. Mason, Alaa El-Din A. Bekhit, and Hina F. Bhat. “Technological, Regulatory, and Ethical Aspects of In Vitro Meat: A Future Slaughter-Free Harvest.” Comprehensive Reviews in Food Science and Food Safety 18, no. 4 (July 2019): 1192-1208. doi.org/10.1111/1541-4337.12473.
Bunge, Jacob. “Cargill Invests in Startup That Grows ‘Clean Meat’ From Cells.” Wall Street Journal, Aug. 23, 2017. Available at wsj.com/articles/cargill-backs-cell-culture-meat-1503486002. Accessed Oct. 20, 2023.
Chen, Lu, Donovan Guttieres, Andrea Koenigsberg, Paul W. Barone, Anthony J. Sinskey, and Stacy L. Springs. “Large-Scale Cultured Meat Production: Trends, Challenges and Promising Biomanufacturing Technologies.” Biomaterials 280 (Jan. 1, 2022): 121274. doi.org/10.1016/j.biomaterials.2021.121274.
Chiorando, Maria. “Over Half Of Young Americans Are ‘Flexitarians’” Plant Based News. Jan. 15, 2021. Available at plantbasednews.org/culture/ethics/over-half-young-americans-flexitarians/. Accessed Oct. 20, 2023
Cho, Dong Seong, and Jason D. Doles. “Preparation of Adipose Progenitor Cells from Mouse Epididymal Adipose Tissues.” Journal of Visualized Experiments: JoVE, no. 162 (Aug. 25, 2020). doi.org/10.3791/61694.
Clugston, Erika. “Mosa Meat: From €250,000 To €9 Burger Patties.” CleanTechnica, Sep. 12, 2019. Available at cleantechnica.com/2019/09/12/mosa-meat-from-e250000-to-e9-burger-patties/. Accessed Oct. 20, 2023.
Cui, H. X., et al. “Method using a co-culture system with high-purity intramuscular preadipocytes and satellite cells from chicken pectoralis major muscle.” Poultry Science 97(10): 3691-3697, Oct. 1, 2018. Doi: 10.3382/ps/pey023
Duckett, Dominic George, Altea Lorenzo-Arribas, Graham Horgan, and Anna Conniff. “Amplification without the Event: The Rise of the Flexitarian.” Journal of Risk Research 24, no. 9 (Nov. 1, 2021): 1049-71. doi.org/10.1080/13669877.2020.1800066.
Fernandez-Cao, Jose Candido, Victoria Arija, Nuria Aranda, Monica Bullo, Josep Basora, Miguel Angel Martinez-Gonzalez, Javier Diez-Espino, and Jordi Salas-Salvad6. “Heme Iron Intake and Risk of New-Onset Diabetes in a Mediterranean Population at High Risk of Cardiovascular Disease: An Observational Cohort Analysis.” BMC Public Health 13 (Nov. 4, 2013): 1042. doi.org/10.1186/1471-2458-13-1042.
Fish, Kyle D., Natalie R. Rubio, Andrew J. Stout, John S. K. Yuen, and David L. Kaplan. “Prospects and Challenges for Cell-Cultured Fat as a Novel Food Ingredient.” Trends in Food Science & Technology 98 (Apr. 1, 2020): 53-67. doi.org/10.1016/j.tifs.2020.02.005.
Godschalk-Broers, Layla, Guido Sala, and Elke Scholten. “Meat Analogues: Relating Structure to Texture and Sensory Perception.” Foods (Basel, Switzerland) 11, no. 15 (Jul. 26, 2022): 2227. doi.org/10.3390/foodsl1152227.
González, Neus, Montse Marqués, Marti Nadal, and José L. Domingo. “Meat Consumption: Which Are the Current Global Risks?A Review of Recent (2010-2020) Evidences.” Food Research International (Ottawa, Ont.) 137 (November 2020): 109341. doi.org/10.1016/j.foodres.2020.109341.
Gstraunthaler, Gerhard. “Alternatives to the Use of Fetal Bovine Serum: Serum-Free Cell Culture.” ALTEX, Vol. 20, No. 4 (2003): 275-81. PMID: 14671707
He, Ya, Qingbin Yuan, Jacques Mathieu, Lauren Stadler, Naomi Senehi, Ruonan Sun, and Pedro J. J. Alvarez. “Antibiotic Resistance Genes from Livestock Waste: Occurrence, Dissemination, and Treatment.” Npj Clean Water 3, no. 1 (Feb. 19, 2020): 1-11. doi.org/10.1038/s41545-020-0051-0.
Henchion, Maeve, Mary McCarthy, Virginia C. Resconi, and Declan Troy. “Meat Consumption: Trends and Quality Matters.” Meat Science, Meat Science, Sustainability & Innovation: ‘60^{th} International Congress of Meat Science and Technology 17-22 Aug. 2014, Punta del Este, Uruguay,’ 98, no. 3 (Nov. 1, 2014): 561-68. doi.org/10.1016/j.meatsci.2014.06.007.
Hendawy, Hanan, Masahiro Kaneda, Elsayed Metwally, Kazumi Shimada, Takashi Tanaka, and Ryou Tanaka. “A Comparative Study of the Effect of Anatomical Site on Multiple Differentiation of Adipose-Derived Stem Cells in Rats.” Cells 10, no. 9 (Sep. 18, 2021): 2469. doi.org/10.3390/cells10092469.
Hu, Frank B., Brett O. Otis, and Gina McCarthy. “Can Plant-Based Meat Alternatives Be Part of a Healthy and Sustainable Diet?” JAMA 322, no. 16 (Oct. 22, 2019): 1547-48. doi.org/10.1001/jama.2019.13187.
Huang, Yin, Dehong Cao, Zeyu Chen, Bo Chen, Jin Li, Jianbing Guo, Qiang Dong, Liangren Liu, and Qiang Wei. “Red and Processed Meat Consumption and Cancer Outcomes: Umbrella Review.” Food Chemistry 356 (Sep. 15, 2021): 129697. doi.org/10.1016/j.foodchem.2021.129697.
Humbird, David. “Scale-up Economics for Cultured Meat.” Biotechnology and Bioengineering 118, no. 8 (August 2021): 3239-50. doi.org/10.1002/bit.27848.
Jones, Bryony A., Delia Grace, Richard Kock, Silvia Alonso, Jonathan Rushton, Mohammed Y. Said, Declan McKeever, et al. “Zoonosis Emergence Linked to Agricultural Intensification and Environmental Change.” Proceedings of the National Academy of Sciences of the United States of America 110, no. 21 (May 21, 2013): 8399-8404. doi.org/10.1073/pnas.1208059110.
Jumabay, Medet. “Dedifferentiated Fat Cells: A Cell Source for Regenerative Medicine.” World Journal of Stem Cells Issue 7, No. 10 (2015): 1202. doi.org/10.4252/wjsc.v7.il0.1202.
   Kolkmann, A. M., M. J. Post, M. a. M. Rutjens, A. L. M. van Essen, and P. Moutsatsou. “Serum-Free Media for the Growth of Primary Bovine Myoblasts.” Cytotechnology 72, no. 1 (February 2020): 111-20. doi.org/10.1007/s10616-019-00361-y.
Li, Xuejie and Li, Jian. “The Flavor of Plant-Based Meat Analogues.” Cereal Foods World, Vol. 65, No. 4, July-August 2020. Doi.org/10.1094/CFW-65-4-0040. Available at cerealsgrains.org/publications/cfw/2020/Documents/CFW-65-4-0040.pdf. Accessed Oct. 20, 2023.
Lu, Xiaodan, Svetlana Altshuler-Keylin, Qiang Wang, Yong Chen, Carlos Henrique Sponton, Kenji Ikeda, Pema Maretich, Takeshi Yoneshiro, and Shingo Kajimura. “Mitophagy Controls Beige Adipocyte Maintenance through a Parkin-Dependent and UCP1-Independent Mechanism.” Science Signaling 11, no. 527 (Apr. 24, 2018): eaap8526. doi.org/10.1126/scisignal.aap8526.
McCormick, Erin. “Eat Just Is Racing to Put ‘No-Kill Meat’ on Your Plate. Is It Too Good to Be True?” The Guardian, Jun. 16, 2021, Available at theguardian.com/food/2021/jun/16/eat-just-no-kill-meat-chicken-josh-tetrick.
Messmer, Tobias, Iva Klevernic, Carolina Furquim, Ekaterina Ovchinnikova, Arin Dogan, Helder Cruz, Mark J. Post, and Joshua E. Flack. “A Serum-Free Media Formulation for Cultured Meat Production Supports Bovine Satellite Cell Differentiation in the Absence of Serum Starvation.” Nature Food 3, no. 1 (January 2022): 74-85. doi.org/10.1038/s43016-021-00419-1.
Michel, Fabienne, Christina Hartmann, and Michael Siegrist. “Consumers' Associations, Perceptions and Acceptance of Meat and Plant-Based Meat Alternatives.” Food Quality and Preference 87 (Jan. 1, 2021): 104063. doi.org/10.1016/j.foodqual.2020.104063.
Nunes, Keith. “Why Are Consumers Buying Fewer Plant-Based Meat Alternatives?” Food Business News. Nov. 17, 2021. Available at foodbusinessnews.net/articles/20067-why-are-consumers-buying-fewer-plant-based-meat-alternatives. Accessed Oct. 20, 2023.
Ong, Wee Kiat, Smarajit Chakraborty, and Shigeki Sugii. “Adipose Tissue: Understanding the Heterogeneity of Stem Cells for Regenerative Medicine.” Biomolecules 11, no. 7 (Jun. 22, 2021): 918. doi.org/10.3390/bioml1070918.
Otte, Joachim, and Ugo Pica-Ciamarra. “Emerging Infectious Zoonotic Diseases: The Neglected Role of Food Animals.” One Health (Amsterdam, Netherlands) 13 (December 2021): 100323. doi.org/10.1016/j.onehlt.2021.100323.
Petrovic, Zoran, Vesna Djordjevic, Dragan Milicevic, Ivan Nastasijevic, and Nenad Parunovic. “Meat Production and Consumption: Environmental Consequences.” Procedia Food Science, The 58^{th} International Meat Industry Conference (MeatCon2015), 5 (Jan. 1, 2015): 235-38. doi.org/10.1016/j.profoo.2015.09.041.
Panella, Stefano, Francesco Muoio, Valentin Jossen, Yves Harder, Regine Eibl-Schindler, and Tiziano Tallone. “Chemically Defined Xeno- and Serum-Free Cell Culture Medium to Grow Human Adipose Stem Cells.” Cells 10, no. 2 (Feb. 22, 2021): 466. doi.org/10.3390/cells10020466.
Pu, Yong, and Almudena Veiga-Lopez. “PPARγ Agonist through the Terminal Differentiation Phase Is Essential for Adipogenic Differentiation of Fetal Ovine Preadipocytes.” Cellular & Molecular Biology Letters 22, no. 1 (December 2017): 6. doi.org/10.1186/si 1658-017-0037-1.
Rogal, Julia, Carina Binder, Elena Kromidas, Julia Roosz, Christopher Probst, Stefan Schneider, Katja Schenke-Layland, and Peter Loskill. “WAT-on-a-Chip Integrating Human Mature White Adipocytes for Mechanistic Research and Pharmaceutical Applications.” Scientific Reports 10, no. 1 (Apr. 20, 2020): 6666. doi.org/10.1038/s41598-020-63710-4.
Rubio, Natalie R., Ning Xiang, and David L. Kaplan. “Plant-Based and Cell-Based Approaches to Meat Production.” Nature Communications 11, no. 1 (Dec. 8, 2020): 6276. doi.org/10.1038/s4l467-020-20061-y.
Santo, Raychel E., Brent F. Kim, Sarah E. Goldman, Jan Dutkiewicz, Erin M. B. Biehl, Martin W. Bloem, Roni A. Neff, and Keeve E. Nachman. “Considering Plant-Based Meat Substitutes and Cell-Based Meats: A Public Health and Food Systems Perspective.” Frontiers in Sustainable Food Systems 4 (2020). doi.org/10.3389/fsufs.2020.00134
Schopow, Nikolas, Sonja Kallendrusch, Siming Gong, Felicitas Rapp, Justus Korfer, Martin Gericke, Nick Spindler, Christoph Josten, Stefan Langer, and Ingo Bechmann. “Examination of Ex-Vivo Viability of Human Adipose Tissue Slice Culture.” Edited by Anna Halama. PLOS ONE 15, no. 5 (May 26, 2020): e0233152. doi.org/10.1371/journal.pone.0233152.
Scipioni, Jade. “This Restaurant Will Be the First Ever to Serve Lab-Grown Chicken (for $23).” CNBC, Dec. 18, 2020. cnbc.com/2020/12/18/singapore-restaurant-first-ever-to-serve-eat-just-lab-grown-chicken.html.
Shah, Mickey, Richard L George, M. Michelle Evancho-Chapman, and Ge Zhang. “Current Challenges in Dedifferentiated Fat Cells Research.” Organogenesis 12, no. 3 (Jul. 2, 2016): 119-27. doi.org/10.1080/15476278.2016.1197461.
Sinke, Pelle and Odegard, Ingrid. “LCA of Cultivated Meat: Future Projections for Different Scenarios.” CE Delft, February 2021, Publication code: 21.190107.019, available at CE_Delft_190107_LCA_of_cultivated_meat_DefPdf Accessed Oct. 20, 2023.
Specht, Liz. “An Analysis of Culture Medium Costs and Production.” The Good Food Institute, Feb. 9, 2020, available at gfi. org/wp-content/uploads/2021/01/clean-meat-production-volume-and-medium-cost.pdf. Accessed Oct. 20, 2023.
Sugihara, H., N. Yonemitsu, S. Miyabara, and K. Yun. “Primary Cultures of Unilocular Fat Cells: Characteristics of Growth in Vitro and Changes in Differentiation Properties.” Differentiation; Research in Biological Diversity 31, no. 1 (1986): 42-49. doi.org/10.1111/j.1432-0436.1986.tb00381.x.
Sugihara, H., N. Yonemitsu, S. Miyabara, and S. Toda. “Proliferation of Unilocular Fat Cells in the Primary Culture.” Journal of Lipid Research 28, no. 9 (September 1987): 1038-45.
Tomiyama, A. Janet, N. Stephanie Kawecki, Daniel L. Rosenfeld, Jennifer A. Jay, Deepak Rajagopal, and Amy C. Rowat. “Bridging the Gap between the Science of Cultured Meat and Public Perceptions.” Trends in Food Science & Technology 104 (Oct. 1, 2020): 144-52. doi.org/10.1016/j.tifs.2020.07.019.
Tso, Rachel, and Ciarin G. Forde. “Unintended Consequences: Nutritional Impact and Potential Pitfalls of Switching from Animal- to Plant-Based Foods.” Nutrients 13, no. 8 (Jul. 23, 2021): 2527. doi.org/10.3390/nul3082527.
Tuomisto, Hanna L., and M. Joost Teixeira de Mattos. “Environmental Impacts of Cultured Meat Production.” Environmental Science & Technology 45, no. 14 (Jul. 15, 2011): 6117-23. doi.org/10.1021/es20013Ou.
Vatanparast, Hassan, Naorin Islam, Mojtaba Shafiee, and D. Dan Ramdath. “Increasing Plant-Based Meat Alternatives and Decreasing Red and Processed Meat in the Diet Differentially Affect the Diet Quality and Nutrient Intakes of Canadians.” Nutrients 12, no. 7 (Jul. 9, 2020): 2034. doi.org/10.3390/nul2072034.
Vliet, Stephan van, James R. Bain, Michael J. Muehlbauer, Frederick D. Provenza, Scott L. Kronberg, Carl F. Pieper, and Kim M. Huffman. “A Metabolomics Comparison of Plant-Based Meat and Grass-Fed Meat Indicates Large Nutritional Differences despite Comparable Nutrition Facts Panels.” Scientific Reports 11, no. 1 (Jul. 5, 2021): 13828. doi.org/10.1038/s41598-021-93100-3.
Waltz, Emily. “Club-Goers Take First Bites of Lab-Made Chicken.” Nature Biotechnology 39, no. 3 (Mar. 1, 2021): 257-58. doi.org/10.1038/s41587-021-00855-1.
Wang, Yaqin, Fabio Tuccillo, Anna-Maija Lampi, Antti Knaapila, Marjo Pulkkinen, Susanna Kariluoto, Rossana Coda, et al. “Flavor Challenges in Extruded Plant-Based Meat Alternatives: A Review.” Comprehensive Reviews in Food Science and Food Safety 21, no. 3 (May 2022): 2898-2929. doi.org/10.1111/1541-4337.12964.
Watson, James E., Niketa A. Patel, Gay Carter, Andrea Moor, Rekha Patel, Tomar Ghansah, Abhishek Mathur, et al. “Comparison of Markers and Functional Attributes of Human Adipose-Derived Stem Cells and Dedifferentiated Adipocyte Cells from Subcutaneous Fat of an Obese Diabetic Donor.” Advances in Wound Care 3, no. 3 (March 2014): 219-28. doi.org/10.1089/wound.2013.0452.
Wei, Shengjuan, Min Du, Zhihua Jiang, Marcio S Duarte, Melinda Fernyhough-Culver, Elke Albrecht, Katja Will, et al. “Bovine Dedifferentiated Adipose Tissue (DFAT) Cells: DFAT Cell Isolation.” Adipocyte 2, no. 3 (Jul. 24, 2013): 148-59. doi.org/10.4161/adip.24589.
Yagi, Ken, Daisuke Kondo, Yasushi Okazaki, and Koichiro Kano. “A Novel Preadipocyte Cell Line Established from Mouse Adult Mature Adipocytes.” Biochemical and Biophysical Research Communications 321, no. 4 (September 2004): 967-74. doi.org/10.1016/j.bbrc.2004.07.055.
Yamanaka, Kumiko, Yuji Haraguchi, Hironobu Takahashi, Ikko Kawashima, and Tatsuya Shimizu. “Development of Serum-Free and Grain-Derived-Nutrient-Free Medium Using Microalga-Derived Nutrients and Mammalian Cell-Secreted Growth Factors for Sustainable Cultured Meat Production.” Scientific Reports 13, no. 1 (Jan. 10, 2023): 498. doi.org/10.1038/s41598-023-27629-w.
Yeun Jr, J.S.K, et al., “Aggregating in vitro-grown adipocytes to produce macroscale cell-cultured fat tissue with tunable lipid compositions for food applications.” Elife. 4: 12:e82120, Apr. 4, 2023. doi: 10.7554/eLife.82120
Zang, Liguo, Suchart Kothan, Yiyi Yang, Xiangyi Zeng, Lingmin Ye, and Jie Pan. “Insulin Negatively Regulates Dedifferentiation of Mouse Adipocytes in Vitro.” Adipocyte 9, no. 1 (Jan. 1, 2020): 24-34. doi.org/10.1080/21623945.2020.1721235.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

All patents, patent applications and publications cited herein are hereby incorporated by reference in their entirety. Those skilled in the art will recognize, or be able to ascertain, using no more than routine experimentation, numerous equivalents to the specific substances and procedures described herein. Such equivalents are considered to be within the scope of this invention and are covered by the following claims.

1. A method of cyclical production of fat cells, the method comprising:
(i) providing a plurality of mature adipocytes;
(ii) incubating the plurality of mature adipocytes under conditions such that at least a portion of the plurality of mature adipocytes dedifferentiates, thereby generating a plurality of dedifferentiated fat cells (DFAT cells);
(iii) culturing the plurality of DFAT cells in a growth medium until a first target biomass is obtained;
(iv) subculturing the first target biomass in one or more subculture populations of DFAT cells; and
(v) incubating the one or more subculture populations of DFAT cells under conditions such that at least a portion of the one or more subculture populations re-differentiates into one or more populations of re-differentiated mature adipocytes.

2. The method of claim 1, further comprising:
(vi) dividing at least one of the one or more populations of re-differentiated mature adipocytes into a first and a second subpopulation;
(vii) incubating the first subpopulation of re-differentiated mature adipocytes under conditions such that at least a portion of the first subpopulation of re-differentiated mature adipocytes dedifferentiates, thereby generating a plurality of second generation DFAT cells; and
(viii) culturing the second subpopulation of re-differentiated mature adipocytes under conditions such that at least a portion of the second subpopulation of re-differentiated mature adipocytes accumulate a target lipid mass.

3. The method of claim 2, wherein the target lipid mass is about 20-90% of the cell volume.

4. The method of claim 2, wherein the second subpopulation of re-differentiated mature adipocytes is cultured in a culture medium comprising glucose at a concentration of at least 4 g/L, optionally, 4 g/L-15 g/L, or 4 g/L-10 g/L, or 4 g/L-8 g/L.

5. The method of claim 1, wherein the one or more subculture populations of DFAT cells are cultured in step v) in a re-differentiation medium comprising one or more components selected from the group consisting of insulin, 3-isobutyl-1-methylxanthine, insulin-transferrin-selenium-X supplement, indomethacin, dexamethasone, rosiglitazone, biotin, pantothenate, a PPARγ agonist, sodium oleate, and wheat germ agglutinin.

6. The method of claim 2, further comprising:
culturing the second generation of DFAT cells in a growth medium until a second target biomass is obtained;
subculturing the second target biomass into a second generation of subcultured DFAT cells; and
incubating the second generation of subcultured DFAT cells under conditions such that at least a portion of the second generation of subcultured DFAT cells re-differentiates into a second generation of re-differentiated mature adipocytes.

7. The method of claim 1, further comprising cyclically repeating one or more of steps ii), iii), iv), or v) for a total of n cycles, wherein the step ii) for each cycle is performed on the re-differentiated mature adipocytes that were re-differentiated in step v) of the immediately preceding cycle,
wherein n is equal to the number of cycles during which the re-differentiated mature adipocytes maintain the ability to proliferate and/or maintain the ability to synthesize and store fat.

8. The method of claim 1, wherein the plurality of mature adipocytes is isolated from adipose cells or from a tissue explant comprising adipose tissue.

9. The method of claim 8, wherein the plurality of mature adipocytes is isolated by:
adding the adipose cells or tissue explant to a digestion buffer in a container;
incubating the adipose cells or tissue explant in the digestion buffer for at least about 30 minutes;
centrifuging the adipose cells and digestion buffer to create a first floating adipocyte layer (FAL) above the digestion buffer and a pelleted tissue at the bottom of the container;
removing the digestion buffer and pelleted tissue from the container;
suspending the first FAL in growth media;
centrifuging the first FAL and growth media to create a second FAL and a second pelleted tissue;
removing the digestion buffer and the second pelleted tissue from the container; and
suspending the second FAL in growth medium, wherein the second FAL comprises the mature adipocytes.

10. The method of claim 9, wherein the adipose cells are added to the digestion buffer at a concentration of about 1 gram of adipose cells or tissue explant per 3 ml of digestion buffer.

11. The method of claim 9, wherein the digestion buffer comprises a collagenase.

12. The method of claim 11, wherein the collagenase is derived from microbial fermentation.

13. The method of claim 11, wherein the collagenase is collagenase IV.

14. The method of claim 13, wherein the digestion buffer comprises between 0.2% and 1% collagenase IV.

15. The method of claim 13, wherein the collagenase IV is from Clostridium histolyticum.

16. The method of claim 8, wherein the tissue explant comprises subcutaneous fat extracted from a subject.

17. The method of claim 16, wherein the subject is a mammal, a bird, a fish, a reptile, a crustacean, or an amphibian.

18. The method of claim 8, wherein the adipose cells do not comprise or were not derived from pluripotent stem cells, embryonic stem cells, or undifferentiated stem cells.

19. The method of claim 8, wherein the adipose cells comprise primary mammalian cells, primary avian cells, primary fish cells, primary reptilian cells, primary crustacean cells, primary amphibian cells, or a combination thereof.

20. The method of claim 9, wherein the centrifuging step is performed without filtration.

21. The method of claim 1, wherein step ii) comprises:
seeding the mature adipocytes in a dedifferentiation container with a volume of growth media sufficient to completely fill the container;
closing the dedifferentiation container to create a hypoxic environment;
inverting the dedifferentiation container;
incubating the inverted dedifferentiation container for a period of time sufficient for the mature adipocytes to adhere to the ceiling surface of the dedifferentiation container; and
allowing the mature adipocytes to dedifferentiate into DFAT cells.

22. The method of claim 21, wherein the inverted dedifferentiation container is incubated for a period of about 10 days.

23. The method of claim 1, wherein the one or more subculture populations of DFAT cells are cultured in step v) under conditions involving the manipulation of one or more parameters to preferentially encourage formation of the re-differentiated mature adipocytes, wherein the one or more parameters comprise: a coating to an interior surface of the container, one or more mechanical properties of a culture environment, growth media density, growth media osmolarity, growth media temperature, factors that encourage DFAT division rather than lipid production, and agents to control formation of non-adipocyte cells.

24. The method of claim 1, wherein the first target biomass is reached upon achieving at least 70% confluence of DFAT cells.

25. The method of claim 1, wherein the growth medium comprises only food-safe components.

26. The method of claim 1, wherein the growth medium is serum-free.

27. The method of claim 26, wherein the growth medium does not comprise fetal bovine serum or fetal calf serum.

28. The method of claim 1, wherein the culturing of step iii) and/or subculturing of step iv) comprises seeding growth medium with a concentration of at least 2000 cells/cm2, and incubating until the cells reach at least 70% confluence.

29. The method of claim 28, wherein the subculturing of step iv) comprises seeding growth medium with a concentration of between about 5,000 cells/cm2 to about 10,000 cells/cm2, and incubating until the cells reach at least 75% confluence.

30. A fat-producing cell line created using the methods of the method of claim 1.

31. The fat-producing cell line of claim 30, wherein the fat-producing cell line comprises a total lipid accumulation of at least 80% of an intracellular volume when the cell line is at least 4 months old.

32. A cell line of re-differentiated fat-producing cells created using the methods of claim 1, wherein the cell line is configured to survive in a suspension culture.

33. An imitation meat product for consumption comprising fat cells created using the methods of claim 1, and an effective amount of an alternative meat source.

34. The imitation meat product of claim 33, wherein the fat cells represent up to about 5% of the imitation meat product.

35. The imitation meat product of claim 33, wherein the alternative meat source comprises cultured meat, a plant-based meat alternative, insect-derived proteins, fermentation-derived products, fungal proteins, or a combination thereof.

36. A method of creating an imitation meat product for consumption comprising:
(i) providing a plurality of mature adipocytes;
(ii) incubating the plurality of mature adipocytes under conditions such that at least a portion of the plurality of mature adipocytes dedifferentiates, thereby generating a plurality of dedifferentiated fat cells (DFAT cells);
(iii) culturing the plurality of DFAT cells in a growth medium until a first target biomass is obtained;
(iv) subculturing the first target biomass in one or more subculture populations of DFAT cells;
(v) incubating the one or more subculture populations of DFAT cells under conditions such that at least a portion of the one or more subculture populations re-differentiates into one or more populations of re-differentiated mature adipocytes; and
(vi) combining the one or more populations of re-differentiated mature adipocytes with an effective amount of an alternative meat source to create the imitation meat product.

37. The method of claim 36, wherein the alternative meat source comprises cultured meat, a plant-based meat alternative, insect-derived proteins, fermentation-derived products, fungal proteins, or a combination thereof.

38. A commercially scalable cell line for producing consumable animal fat, the cell line comprising re-differentiated fat-producing cells created by the methods of of claim 1, wherein the cell line is produced in a commercial-scale bioreactor.

39. A method of creating at least two distinct populations of dedifferentiated fat (DFAT) cells, the method comprising:
(i) obtaining adipose cells or a tissue explant that comprises adipose tissue;
(ii) isolating a population of mature adipocytes from the adipose cells or tissue explant, wherein isolating the population of mature adipocytes comprises:
a) adding the adipose cells or tissue explant to a digestion buffer in a container, wherein the digestion buffer comprises a collagenase;
b) incubating the adipose cells or tissue explant in the digestion buffer until homogeneous digestion is achieved, optionally, at least about 30 minutes;
c) centrifuging the adipose cells and digestion buffer to create a first floating adipocyte layer (FAL) above the digestion buffer and a pelleted tissue at the bottom of the container, wherein this step of centrifuging does not comprise filtration;
d) removing the digestion buffer and pelleted tissue from the container;
e) suspending the first FAL in growth media;
f) centrifuging the first FAL and growth media to create a second FAL and a second pelleted tissue;
g) removing the digestion buffer and the second pelleted tissue from the container; and
h) suspending the second FAL in growth media, wherein the second FAL comprises the mature adipocytes;
(iii) seeding the mature adipocytes in a dedifferentiation container with a volume of growth medium sufficient to completely fill the container;
(iv) closing the dedifferentiation container to create a hypoxic environment;
(v) inverting the dedifferentiation container;
(vi) incubating the inverted dedifferentiation container for a period of time sufficient for the mature adipocytes to adhere to a celling surface of the dedifferentiation container;
(vii) allowing the mature adipocytes to dedifferentiate into DFAT cells; and
(viii) establishing cell lines derived from two or more of the DFAT cells generated in step vii).

40. The method of claim 39, wherein the period of time for which the dedifferentiation container is incubated is about 10 hours.

41. The method of claim 39, wherein the collagenase is derived from microbial fermentation.

42. The method of claim 39, wherein the collagenase comprises collagenase IV.

43. The method of claim 39, wherein the digestion buffer comprises between 0.2% and 1% collagenase IV.

44. The method of claim 42, wherein the collagenase IV is from Clostridium histolyticum.

45. The method of claim 39, wherein the tissue explant comprises subcutaneous fat extracted from a subject.

46. The method of claim 45, wherein the subject comprises a mammal, a bird, a fish, a reptile, a crustacean, or an amphibian.

47. The method of claim 45, wherein the adipose cells do not comprise or were not derived from pluripotent stem cells, embryonic stem cells, or undifferentiated stem cells.

48. The method of claim 45, wherein the adipose cells comprise primary mammalian cells, primary avian cells, primary fish cells, primary reptilian cells, primary crustacean cells, primary amphibian cells, or a combination thereof.

49. A method of obtaining avian dedifferentiated fat (DFAT) cells, the method comprising:
i) providing adipose cells or a tissue explant comprising adipose tissue from an avian source;
ii) introducing the adipose cells or tissue explant to a digestion buffer in a container, wherein the digestion buffer comprises collagenase IV;
iii) incubating the adipose cells or tissue explant in the digestion buffer for at least about 30 minutes;
iv) centrifuging the adipose cells and digestion buffer to create a first floating adipocyte layer (FAL) above the digestion buffer and a pelleted tissue at the bottom of the container;
v) removing the digestion buffer and pelleted tissue from the container;
vi) suspending the first FAL in growth media;
vii) centrifuging the first FAL and growth media to create a second FAL and a second pelleted tissue;
viii) removing the digestion buffer and the second pelleted tissue from the container;
ix) suspending the second FAL in growth medium, wherein the second FAL comprises the mature adipocytes;
x) seeding the mature adipocytes in a dedifferentiation container with a volume of growth medium sufficient to completely fill the container;
xi) closing the dedifferentiation container to create a hypoxic environment;
xii) inverting the dedifferentiation container;
xiii) incubating the inverted dedifferentiation container for a period of time sufficient for the mature adipocytes to adhere to the ceiling surface of the dedifferentiation container; and
xiv) allowing the mature adipocytes to dedifferentiate into DFAT cells.

50. The method of claim 49, wherein the adipose cells or tissue explant are added to the digestion buffer at a concentration of about 1 gram of adipose cells or tissue explant per 3 ml of digestion buffer.

51. The method of claim 49, wherein the collagenase IV is derived from microbial fermentation.

52. The method of claim 49, wherein the digestion buffer comprises between about 0.2% and about 1% collagenase IV.

53. The method of claim 49, wherein the collagenase IV is from Clostridium histolyticum.

54. The method of claim 49, wherein the avian source is a chicken, turkey, duck, pigeon, guinea fowl, or ostrich.

55. The method of claim 49, wherein the adipose cells do not comprise or were not derived from pluripotent stem cells, embryonic stem cells, or undifferentiated stem cells.

56. The method of claim 49, wherein the adipose cells comprise primary avian cells.

57. The method of claim 49, wherein the centrifuging step is performed without filtration.

58. The method of claim 49, wherein the period of time sufficient for the mature adipocytes to adhere to the ceiling surface of the dedifferentiation container is about 10 days.

59. The method of claim 49, wherein the digestion buffer does not comprise collagenase I or collagenase II.
